(19) United States
(12) Patent Application Publication
(10) Pub. No.: US 2007/0020759 A1
(43) Pub. Date: Jan. 25, 2007

(54) Therapeutic reprogramming of germ line stem cells

(75) Inventors: Chauncey B. Sayre, Irvine, CA (US); Francisco J. Silva, Rancho Cucamonga, CA (US); Kwok-Yuen F. Pau, Irvine, CA (US)

Correspondence Address: PRESTON GATES & ELLIS LLP, 1900 MAIN STREET, SUITE 600, IRVINE, CA, 92614-7319, US

(73) Assignee: PrimeGen Biotech LLC, Irvine, CA (US)

(21) Appl. No.: 11/488,362

(22) Filed: Jul. 17, 2006

Related U.S. Application Data
(63) Continuation of application No. 11/279,611, filed on Apr. 13, 2006, now abandoned.
(63) Continuation-in-part of application No. 11/060,131, filed on Feb. 16, 2005.
(60) Provisional application No. 60/699,680, filed on Jul. 15, 2005.
(60) Provisional application No. 60/671,826, filed on Apr. 14, 2005.
(60) Provisional application No. 60/588,146, filed on Jul. 15, 2004.

(51) Int. Cl. C12N 5/08; C12N 15/09

(52) U.S. Cl. 435/366; 435/455; 435/404

(57) ABSTRACT

Pluripotent therapeutically programmed cells and methods for making such cells are provided. The pluripotent therapeutically programmed cells are post-natal stem cells which have been matured such that they represent either a more differentiated state or a less differentiated state after contact with stimulatory factors. The pluripotent therapeutically reprogrammed cells are suitable for cellular regenerative therapy and have the potential to differentiate into more committed cell lineages. Also disclosed are culture media for therapeutically reprogramming post-natal stem cells.

### CROSS REFERENCE TO RELATED APPLICATIONS

This application claims the benefit under 35 U.S.C. §119(e) of United States Provisional Patent Application No. 60/699,680 filed Jul. 15, 2005 and is a continuation-in-part of U.S. patent application Ser. No. 11/279,611 filed Apr. 13, 2006, which claims the benefit under 35 U.S.C. §119(e) of Provisional Patent Application No. 60/671,826 filed Apr. 15, 2005. The present application is also a continuation-in-part of U.S. patent application Ser. No. 11/060,131 filed Feb. 16, 2005 which claims the benefit under 35 U.S.C. §119(e) of Provisional Patent Application No. 60/588,146 filed Jul. 15, 2004. All the above-referenced applications are incorporated by reference herein in their entirety.

### FIELD OF THE INVENTION

The present invention relates to the field of therapeutically reprogrammed cells. Specifically, therapeutically reprogrammed cells are provided that are not compromised by the aging process, are immunocompatible and will function in the appropriate post-natal cellular environment to yield functional cells after transplantation.

### BACKGROUND OF THE INVENTION

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types (Kanatsu-Shinohara M. et al. Generation of pluripotent stem cells from neonatal mouse testis. Cell 119:1001-12, 2004).s

Stem cells are a rare population of cells that can give rise to vast range of cells tissue types necessary for organ maintenance and function. These cells are defined as undifferentiated cells that have two fundamental characteristics; (i) they have the capacity of self-renewal, (ii) they also have the ability to differentiate into one or more specialized cell types with mature phenotypes. There are three main groups of stem cells; (i) adult or somatic (post-natal), which exist in all post-natal organisms, (ii) embryonic, which can be derived from a pre-embryonic or embryonic developmental stage and (iii) fetal stem cells (pre-natal), which can be isolated from the developing fetus. Each group of stem cells has their own advantages and disadvantages for cellular regeneration therapy, specifically in their differentiation potential and ability to engraft and function de novo in the appropriate or targeted cellular environment.

In the post-natal animal there are cells that are lineage-committed progenitor stem cells and lineage-uncommitted pluripotent stem cells, which reside in connective tissues providing the post-natal organism the cells required for continual organ or organ system maintenance and repair. These cells are termed somatic or adult stem cells and can be quiescent or non-quiescent. Typically adult stem cells share two characteristics: (i) they can make identical copies of themselves for long periods of time (long-term self renewal); and (ii) they can give rise to mature cell types that have characteristic morphologies and specialized functions.

Much of the understanding of stem cell biology has been derived from hematopoietic stem cells and their behavior after bone marrow transplantation. There are several types of adult stem cells within the bone marrow niche, each having unique properties and variable differentiation ability in relation to their cellular environment. Somatic stem cells isolated from human bone marrow transferred in utero into pre-immune sheep fetuses have the ability to xenograft into multiple tissues. Also within the bone marrow niche are mesenchymal stem cells, which have a wide range of non-hematopoietic differentiation abilities, including bone, cartilage, adipose, tendon, lung, muscle, marrow stroma, and brain tissues. In addition, neural stem cells, pancreatic, muscle, adipose, ovarian and spermatogonial stem cells have been found. The therapeutic utility of somatic or post-natal stem cells has been demonstrated and realized through the use of bone marrow transplants. However, adult somatic stem cells have genomes that have been altered by aging and cell division. Aging results in an accumulation of free radical insults, or oxidative damage, that can predispose the cell to forming neoplasms, reduce cell differentiation ability or induce apoptosis. Repeated cell division is directly related to telomere shortening which is the ultimate cellular clock that determines a cells functional life-span. Consequently, adult somatic stem cells have genomes that have sufficiently diverged from the physiological prime state found in embryonic and prenatal stem cells.

Unfortunately, virtually every somatic cell in the adult animal's body, including stem cells, possess a genome ravaged by time and repeated cell division. Thus until now the only means for obtaining stem cells having an undamaged, or prime state physiological genome, was to recover stem cells from aborted embryos or embryos formed using in vitro fertilization techniques. However, scientific and ethical considerations have slowed the progress of stem cell research using embryonic stem cells. Generation of embryonic stem cell lines had been thought to provide a renewable source of embryonic stem cells for both research and therapy but recent reports indicate that existing cell lines have been contaminated with immunogenic animal molecules (Martin M. et al., Human embryonic stem cells express an immunogenic nonhuman sialic acid. Nature Medicine 11:228-32, 2005).

Another problem associated with using adult stems cells is that these cells are not immunologically privileged, or can lose their immunological privilege after transplant. (The term “immunologically privileged” is used to denote a state where the recipient's immune system does not recognize the cells as foreign). Thus, only autologous transplants are possible in most cases when adult stem cells are used. Thus, most presently envisioned forms of stem cell therapy are essentially customized medical procedures and therefore economic factors associated with such procedures limit their wide ranging potential. Additional barriers to the use of currently available

Moreover, stem cells must be induced to mature into the organ or cell type desired to be useful as therapeutics. The factors affecting stem cell maturation in vivo are poorly understood and even less well understood ex vivo. Thus, present maturation technology relies on serendipity and biological processes largely beyond the control of the administering scientist or recipient.

Current research is focused on developing embryonic stem cells as a source of totipotent or pluripotent immunologically privileged cells for use in cellular regenerative therapy. However, since embryonic stem cells themselves may not be appropriate for direct transplant as they form teratomas after transplant, they are proposed as “universal donor” cells that can be differentiated into customized pluripotent, multipotent or committed cells that are appropriate for transplant. Additionally there are moral and ethical issues associated with the isolation of embryonic stem cells from human embryos.

Generation of pluripotent cell lines which can be safely used in regenerative medicine has a great impact in biotechnology. In this regard, embryonic stem (ES) cells can become a potential cell source for cell replacement therapy because they can be propagated indefinitely and differentiated into phenotypes of all three germ layers. However, before ES cell applications can be realized, the ethical issue must be resolved, and the formation of teratomas after transplantation of ES cells or their derivative must be overcome. Adult stem cells originated from various tissues are considered as alternative sources for cell-based therapy because they do not form teratomas after transplantation, and they maintain the plasticity to differentiate into phenotypes of the same lineage. They can even be induced to trans-differentiate into cell types of a different lineage, or reprogrammed to become pluripotent stem cells for clinical applications. Among all adult stem cells, only germ line stem cells (GSC) retain the ability to transmit genetic information to offspring. There are also several lines of evidences suggesting that GSCs acquire pluripotentiality through reprogramming processes during normal development. Therefore, GSCs are considered as an excellent adult stem cell model for generation of pluripotent cell lines for therapeutic purposes. Recently, ES cell-like cells have been generated from neonatal mouse testes. These cells exhibit similar molecular and functional characteristics as ES cells and generate teratomas when injected into the testis of immune compromised mice. However, these ES-like cells can not be generated from the adult testis, reducing their value for therapeutic purposes.

Therefore, there is a need for sources of biologically useful, pluripotent stem cells having genomes in a nearly physiologically prime state. Furthermore, there is a need for sources of biologically useful, pluripotent stem cells having genomes in a nearly physiologically prime state that maintain their immunological privilege in recipients for a time period sufficient to be therapeutically useful. Additionally, there is a need to condition stem cell transplants either in vivo or ex vivo in order to maximize the potential that the transplanted stem cell will mature into the intended tissue.

### SUMMARY OF THE INVENTION

The present invention provides biologically useful pluripotent therapeutically reprogrammed human cells, generated from human post-natal stem cells, having minimal oxidative damage and telomere lengths that compare favorably with the telomere lengths of undamaged, pre-natal or embryonic stem cells (that is, the therapeutically reprogrammed human cells of the present invention possess near prime physiological state genomes). Moreover the therapeutically reprogrammed human cells of the present invention are immunologically privileged and therefore suitable for therapeutic applications. Furthermore, the present invention includes related methods for maturing human post-natal stem cells made in accordance with the teachings of the present invention into specific host tissues.

In an embodiment of the present invention, a cell culture medium for therapeutically reprogramming human stem cells is provided wherein the cell culture medium comprises a cell culture growth medium base; a plurality of vitamins and minerals and a plurality of cell growth and maturation factors.

In another embodiment of the present invention, the cell culture medium is serum free and is designated PM-10, PM-20 or PM-100; the plurality of cell growth and maturation factors comprise factors selected from the group consisting of recombinant human epidermal growth factor, recombinant human fibroblast growth factor 2, recombinant human glial cell derived neurotrophic factor and human leukemia inhibitory factor. The recombinant human epidermal growth factor is present at a concentration of between approximately 10 ng/mL and approximately 40 ng/mL, preferably approximately 20 ng/mL. The recombinant human fibroblast growth factor 2 is present at a concentration of between approximately 1 ng/mL and approximately 120 ng/mL. The recombinant human glial cell derived neurotrophic factor is present at a concentration of between approximately 2 ng/mL and approximately 40 ng/mL, preferably between approximately 10 ng/mL (PM-10 and PM-100) and approximately 20 ng/mL (PM-20). The human leukemia inhibitory factor is present at a concentration of between approximately 1,000 units/mL and approximately 10,000 units/mL, preferably approximately 1,000 units/mL.

In another embodiment of the present invention, the cell culture medium contains low concentrations of serum and or serum replacement and is designated PM-1, PM-5 or PM-101; the serum component is human serum or fetal bovine serum at a concentration of between approximately 1 % and approximately 5%, preferably approximately 1 %. The plurality of cell growth and maturation factors comprise factors selected from the group consisting of recombinant human epidermal growth factor, recombinant human fibroblast growth factor 2, recombinant human glial cell derived neurotrophic factor and murine leukemia inhibitory factor. The recombinant human epidermal growth factor is present at a concentration of between approximately 10 ng/mL and approximately 40 ng/mL, preferably approximately 20 ng/mL. The recombinant human fibroblast growth factor 2 is present at a concentration of between approximately 10 ng/mL and approximately 120 ng/mL. The recombinant human glial cell derived neurotrophic factor is present at a concentration of between approximately 2 ng/mL and approximately 40 ng/mL, preferably approximately 2 ng/mL. The murine leukemia inhibitory factor is present at a concentration of between approximately 1,000 units/mL and approximately 10,000 units/mL, preferably approximately 1,000 units/mL.

In an embodiment of the present invention, the cell culture medium contains high concentration of serum or serum replacement and is designated PM-3; the serum component is human serum or fetal bovine serum at a concentration of between approximately 10% and approximately 15%, preferably approximately 12%. The plurality of cell growth and maturation factors comprise factors selected from the group consisting of recombinant human epidermal growth factor, recombinant human fibroblast growth factor 2, recombinant human glial cell derived neurotrophic factor and murine leukemia inhibitory factor. The recombinant human epidermal growth factor is present at a concentration of between approximately 10 ng/mL and approximately 40 ng/mL, preferably approximately 20 ng/mL. The recombinant human fibroblast growth factor 2 is present at a concentration of between approximately 10 ng/mL and approximately 40 ng/mL, preferably approximately 10 ng/mL. The recombinant human glial cell derived neurotrophic factor is present at a concentration of between approximately 2 ng/mL and approximately 40 ng/mL, preferably approximately 2 ng/mL. The murine leukemia inhibitory factor is present at a concentration of between approximately 1,000 units/mL and approximately 10,000 units/mL, preferably approximately 1,000 units/mL.

In another embodiment of the present invention, the cell culture medium is designated Expansion Medium; the serum component is human serum or fetal bovine serum or serum replacement at a concentration of between approximately 12% and approximately 17%, preferably approximately 15%. The plurality of cell growth and maturation factors comprise factors selected from the group consisting of recombinant human epidermal growth factor, recombinant human fibroblast growth factor 2, recombinant human glial cell derived neurotrophic factor and murine leukemia inhibitory factor. The murine leukemia inhibitory factor is present at a concentration of between approximately 1,000 units/mL and approximately 10,000 units/mL, preferably approximately 1,000 units/mL.

In an embodiment of the present invention, a therapeutic reprogramming method is provided comprising: isolating a stem cell; contacting the stem cell with a medium comprising stimulatory factors which induce development of the stem cell into a therapeutically reprogrammed cell; recovering the therapeutically reprogrammed cell from the medium; and implanting the therapeutically reprogrammed cell, or a cell matured therefrom, into a host in need of a therapeutically reprogrammed cell.

In an embodiment of the therapeutic reprogramming method of the present invention, the stem cell is a primordial sex cell, for example a spermatogonial stem cell.

In an embodiment of the therapeutic reprogramming method of the present invention, the stem cell is a human post-natal stem cell such as a primordial sex cell. In another embodiment the primordial sex cell is a diploid germ cell isolated from a male or female post-natal source. In another embodiment, the primordial sex cell is a spermatogonial stem cell.

In another embodiment of the therapeutic reprogramming method of the present invention the medium comprises PM-1, PM-3, PM-5 or PM-101 medium or Expansion Medium.

In another embodiment of the therapeutic reprogramming method of the present invention the medium comprises PM-10, PM-20 or PM-100 medium.

In an embodiment of the therapeutic reprogramming method of the present invention the cell matured from the therapeutically reprogrammed human cell is a cardiac myocyte.

In another embodiment of the therapeutic reprogramming method of the present invention, the cell matured from the therapeutically reprogrammed human cell is a glial cell.

In another embodiment of the present invention, a cell culture media for therapeutic reprogramming of stem cells designated PM-1 is provided consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL recombinant human epidermal growth factor, 10 ng/mL recombinant human fibroblast growth factor 2, 2 ng/mL recombinant human glial cell derived neurotrophic factor and 1,000 units murine leukemia inhibitory factor.

In yet another embodiment of the present invention, a cell culture media for therapeutic reprogramming of stem cells designated PM-3 is provided consisting essentially of Dulbecco's Modified Eagle's Medium, 2 mM L-glutamine, 0.1 mM MEM non-essential amino acids, 1× penicillin/streptomycin, 0.1 mM β-mercaptoethanol, the improvement comprising 12% fetal bovine serum, 20 ng/mL recombinant human epidermal growth factor, 10 ng/mL recombinant human fibroblast growth factor 2, 2 ng/mL recombinant human glial cell derived neurotrophic factor and 1,000 units murine leukemia inhibitory factor.

In another embodiment of the present invention, a cell culture media for therapeutic reprogramming of stem cells designated Expansion Medium is provided consisting essentially of Dulbecco's Modified Eagle's Medium, 2 mM L-glutamine, 0.1 mM MEM non-essential amino acids, 1× penicillin/streptomycin, 0.1 mM β-mercaptoethanol, the improvement comprising 15% fetal bovine serum and 1,000 units murine leukemia inhibitory factor.

In another embodiment of the present invention, a cell culture media for therapeutic reprogramming of stem cells designated PM-10 is provided consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 20 ng/mL recombinant human epidermal growth factor, 1 ng/mL recombinant human fibroblast growth factor 2, 10 ng/mL recombinant human glial cell derived neurotrophic factor and 1,000 units/mL human leukemia inhibitory factor.

In another embodiment of the present invention, a cell culture media for therapeutic reprogramming of human post-natal stem cells designated PM-20 is provided consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL recombinant human epidermal growth factor, 1 ng/mL recombinant human fibroblast growth factor 2, 20 ng/mL recombinant human glial cell derived neurotrophic factor and 1,000 units human leukemia inhibitory factor.

In another embodiment of the present invention, a cell culture media for therapeutic reprogramming of stem cells designated PM-100 is provided consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 20 ng/mL recombinant human epidermal growth factor, 110 ng/mL recombinant human fibroblast growth factor 2, 10 ng/mL recombinant human glial cell derived neurotrophic factor and 1,000 units/mL human leukemia inhibitory factor.

In another embodiment of the present invention, a cell culture media for therapeutic reprogramming of human post-natal stem cells designated PM-5 is provided consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 ug/mL insulin, 1× penicillin/streptomycin, 10×10⁻⁵ M β-mercaptoethanol, the improvement comprising 1% fetal bovine serum, 20 ng/mL recombinant human epidermal growth factor, 2 ng/mL recombinant human fibroblast growth factor 2, 20 ng/mL recombinant human glial cell derived neurotrophic factor and 1,000 units human leukemia inhibitory factor.

In another embodiment of the present invention, a cell culture media for therapeutic reprogramming of human post-natal stem cells designated PM-101 is provided consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL recombinant human epidermal growth factor, 10 ng/mL recombinant human fibroblast growth factor 2, 10 ng/mL recombinant human glial cell derived neurotrophic factor and 1,000 units human leukemia inhibitory factor.

In one embodiment of the present invention, a therapeutic reprogramming method is provided comprising isolating a stem cell; contacting the stem cell with a medium comprising stimulatory factors which induce development of the stem cell into a therapeutically reprogrammed cell; and recovering the therapeutically reprogrammed cell from the medium.

In another embodiment of the therapeutic reprogramming method of the present invention, the stem cell is a primordial sex cell. In yet another embodiment, the primordial sex cell is a spermatogonial stem cell.

In yet another embodiment of the therapeutic reprogramming method of the present invention, the medium comprises PM-1 medium. In another embodiment, the medium comprises PM-3 medium. In another embodiment, the medium comprises Expansion Medium.

In another embodiment of the therapeutic reprogramming method of the present invention, the therapeutically reprogrammed cell is a pluripotent germ stem cell. In another embodiment, the cell matured from the therapeutically reprogrammed cell is a cardiac myocyte.

In another embodiment of the therapeutic reprogramming method of the present invention, the stem cell expresses c-kit.

In another embodiment of the therapeutic reprogramming method of the present invention, the method further comprises culturing said pluripotent germ stem cell.

In yet another embodiment of the therapeutic reprogramming method of the present invention, the method further comprises implanting the therapeutically reprogrammed cell, or a cell matured therefrom, into a host in need of a therapeutically reprogrammed cell.

In an embodiment of the present invention, a pluripotent therapeutic composition is provided comprising a therapeutically reprogrammed human post-natal stem cell. In another embodiment, the human post-natal stem cell is a primordial sex cell. In another embodiment, the primordial sex cell is a diploid germ cell isolated from a male or female post-natal source. In yet another embodiment, the primordial sex cell is a spermatogonial stem cell.

In another embodiment of the present invention, a pluripotent therapeutic composition is provided comprising a therapeutically reprogrammed human post-natal stem cell and wherein the therapeutically reprogrammed human post-natal stem cell is produced according to the therapeutic reprogramming method of the present invention.

### BRIEF DESCRIPTION OF THE FIGURES

FIG. 1 depicts therapeutic reprogramming of SSCs to pluripotent germ stem (PGS) cells according to the teachings of the present invention. Shortly after culture, down-regulation of Oct-4 was observed (A-B). After the attachment of the cells in the second week of culture obvious morphological changes occurred (C-D). Approximately three weeks after culture, colonies containing small round cells were formed (E). Up-regulation of Oct-4, as indicated by the expression of GFP, was observed about one month after culture (F). Images of three different PGS Cell lines (PG-mGSC1-2, neonatal OG2 and PG-mGSC-3 adult OG2) are presented in panels G-I. Scale bars: 70 μm.

FIG. 2 depicts isolation of c-kit positive and c-kit negative subpopulations from mouse testicular stem cells using flow cytometry according to the teachings of the present invention. Oct-4 positive cells as detected by GFP expression were found as a distinct cell population compared to wild type testicular cells (A-B). Among the Oct-4 positive cells, two subpopulations consisting of c-kit positive (R5) and c-kit negative cells (R2) were found (C-D). The 580 channel (AutoFI) was used to gate out auto-fluorescent cells.

FIG. 3 depicts immunolocalization of pluripotent markers in PGS colonies according to the teachings of the present invention. A: Oct-4; B: Nanog; C: SSEA-1; D: Alkaline phosphatase. Scale bars: A: 20 μm; B and D: 100 μm; C: 10 μm.

FIG. 4 depicts expression of pluripotent marker genes in PGS colonies made according to the teachings of the present invention as detected by RT-PCR. Lane 1: water, Lane 2: mouse testis, Lane 3: mouse ES cells, Lanes 4-6: PGS lines 1-3.

FIG. 5 depicts the comparison of SSEA-1 expression between PGS and ES cells made according to the teachings of the present invention using flow cytometry. Both the percentage of SSEA-1 positive cells and the intensity of SSEA-1 staining are lower in the PGS cells than the ES cells.

FIG. 6 depicts telomerase activity of a PGS cell line made according to the teachings of the present invention as compared to adult adipose stem cells and mouse ES cells. Lane 1: heat-inactivated mouse adipose derived stem cells (mADSC); 2: intact mADSC; 3: heat-inactivated and sorted mouse ESC; 4: sorted mouse ES cells; 5: sorted c-kit positive cells, heat inactivated; 6: sorted c-kit positive cells; 7: sorted c-kit negative cells, heat inactivated; 8: sorted c-kit negative cells; 9: PGS cells (passage 10), heat inactivated; 10: PGS cells (passage 10): 11: cell extract lysis buffer only; 12: low molecular weight ladder.

FIG. 7 depicts the karyotype of a PGS cell line made according to the teachings of the present invention. The karyotype is representative of 80 metaphase spreads analyzed. After 15 passages, the cells exhibit normal karyotype.

FIG. 8 depicts amplification of GFP DNA in tissues of the chimeric pups generated from PGS cells made according to the teachings of the present invention using PCR. No GFP was observed in reactions lacking template DNA as well as DNA collected from CD1 mouse used as negative controls. Tissue samples from chimeric pups generated from GFP positive ES cells were used as positive control.

FIG. 9 depicts incorporation of PGS cells made according to the teachings of the present invention into the inner cell mass after blastocyst injection. The image, which showed GFP signals, was taken 24 hour after injection into CD-1 recipient blastocysts. Scale bar: 25 μm.

FIG. 10 depicts formation of teratomas and regeneration of spermatogenesis after testicular transplantation of freshly isolated GFP positive cells (B), sorted c-kit positive cells (C), sorted c-kit negative cells (D), PGS cell line (E) and mouse ES cells (F) according to the teachings of the present invention. A; represents the histology of the control testis 2.5 months after busulfan treatment. The majority of seminiferous tubules are depleted from endogenous spermatogenesis. Among the cells transplanted, only the ES cells formed teratomas (F), and only the freshly isolated GFP positive cells and the c-kit negative cells regenerated spermatogenesis, as evaluated six weeks after transplantation (B, D). Scale bars: A, C, E, F: 300 μm; B and D: 175 μm.

FIG. 11 depicts in vitro differentiation of PGS cells into cardiomyocytes and adipocytes according to the teachings of the present invention. Under some culture conditions germ line stem cells formed aggregates (A). Within 1-2 days, these aggregates continued to differentiate into cell layers (B). Both aggregates and cell layers expressed cardiomyocyte markers troponin-1 (D and E). Moreover, some aggregates exhibited rhythmic contractions (C). Germ line stem cells also spontaneously differentiated into adipocytes as shown with Oil Red staining (F). RT-PCR analysis showed the expression of cardiac specific genes NKx-2.5, and GATA-4 in PGS cells after spontaneously differentiation into cardiomyocytes (G). Scale bars: A, C, D: 60 μm; B and E: 90 μm; F: 12 μm.

FIG. 12 depicts induced differentiation of PGS cells into neurons and cardiomyocytes according to the teachings of the present invention. PGS cells formed embryoid bodies (EBs) 48 hr after culture in non-adhesive culture plates. Cells within the aggregates were still GFP positive indicating their Oct-4 expression (A). At day 4 of aggregation, all the EBs lost their GFP expression, indicating their differentiation (B). Two weeks after differentiation induction, nestin positive neuroprogenitor cells (C), GFAP positive astrocytes (D) and neurofilament positive cells (E) were found. Aggregated PGS cells directed to cardiomyocytes using AZA and Cariogenol-C (F-G). Shortly after induction, cells expressing cardiomyocyte specific troponin were formed (F). Cardiac specific myosin heavy chain positive cells are shown in the monolayer of differentiated PGS cells (G). Scale bars: A, B, F, G: 45 μm; C-E: 25 μm.

FIG. 13 depicts expression of neural and cardiac gene markers after differentiation induction (A, B) according to the teachings of the present invention. Note the presence of neuroprogenitor marker nestin and the expression of neuronal markers MAP-2 and GFAP in the differentiated PGS cells as well as ES cells which have gone through the same differentiation protocol (A). Expression of cardiac specific gene NKx-2.5 in PGS cells after differentiation with aza, cardiogenol-C or combination (B).

FIG. 14 depicts fluorescence-activated cell sorting for enrichment of human germ-line stem cells with antibodies to stem cell markers Oct-4, SSEA-4, Tra-1-81 and Tra-1 -60.

FIG. 15 depicts human germ-line stem cells isolated by a one enzyme (FIG. 15A) or three enzyme (FIG. 15B) method after the adherence step of the therapeutic reprogramming method of the present invention.

FIG. 16 depicts the attachment of human germ-line stem cells in the therapeutic reprogramming method of the present invention.

FIG. 17 depicts human germ-line stem cells in “egg-like” formation in the therapeutic reprogramming method of the present invention.

FIG. 18 depicts human germ-line stem cells in “grape-like” clusters during the therapeutic reprogramming method of the present invention.

FIG. 19 depicts colonies of human germ-line stem cells in Step 3 of the PM-10/PM-20 therapeutic reprogramming method of the present invention.

FIG. 20 depicts colonies of human germ-line stem cells according to the teachings of the therapeutic reprogramming method of the present invention.

FIG. 21 depicts therapeutically reprogrammed pluripotent human germ-line stem (PGS) cells according to the teachings of the present invention stained with anti-human mitochondrial antibody.

FIG. 22 depicts therapeutically reprogrammed pluripotent human stem cells according to the teachings of the present invention stained with anti-human Oct-4 (FIG. 22A) or anti-human Nanog (FIG. 22B) antibody.

FIG. 23 depicts therapeutically reprogrammed pluripotent human stem cells according to the teachings of the present invention having up-regulated Oct-4 and Nanog after 9 days of reprogramming in the PM-10 medium. Adult human testicular tissues (lane 3) and the isolated testicular stem cells (day 0, lane 4) do not express pluripotent genes, particularly Oct-4. The testicular stem cells express the germ-line specific marker DAZL.

FIG. 24A-B depicts therapeutically reprogrammed pluripotent human stem cells 11 days (FIG. 24A) and 22 days (FIG. 24B) into the cardiac differentiation process according to the teachings of the present invention.

FIG. 25A-G depicts therapeutically reprogrammed pluripotent human stem cells which have differentiated into cardiac cells according to the teachings of the present invention evidenced by staining with antibodies to the cardiac cell marker cardiac troponin (FIG. 25A-C), alpha actin (FIG. 25D), desmin (FIG. 25E), cardiac myosin (FIG. 25F), and by the expression of cardiac-specific gene markers, including Nkx2.5, GATA-4 and alpha actin (FIG. 25G)

FIG. 26A-E depicts therapeutically reprogrammed pluripotent human stem cells which have differentiated into neural cells by different culturing protocols according to the teachings of the present invention. Astroglial cells were induced by a medium (PM-SHH, based either on PM-1 or PM-10) containing the neural differentiation growth factor, sonic hedgehog (SHH), as evidenced by staining with antibodies to the glial cell marker glial fibrillary acidic protein (GFAP) (FIG. 26A). A control medium (PM-1) without the addition of SHH did not induce glial cell differentiation (FIG. 26B). Neuronal cells were also induced by PM-SHH as evidenced by the expression of neuronal gene markers, neurofilament-68 and GAD-67 (FIG. 26C). Oligodendroglial cells were induced by replacing the PM-10 medium with a DMEM/F12-based medium containing differentiation factors, including SHH, FGF8 and BDNF and evidenced by staining with antibodies to myelin basic protein (MBP, FIG. 26D) and galactocerebroside-C (Gal-C, FIG. 26E).

FIG. 27A-F depicts therapeutically reprogrammed pluripotent human stem cells which have differentiated into chondrocytes according to the teachings of the present invention by culturing in chondrogenic SingleQuots® medium containing TGF-3β as evidenced by staining with alcian blue (FIG. 27A), expression of chondrocyte markers (FIG. 27B). Additionally, therapeutically reprogrammed pluripotent human stem cells were differentiated into osteocytes according to the teachings of the present invention by culturing in DMEM-LG/GL medium containing dexamethasone, ascorbic acid and B-glycerophosphate as evidenced by staining with alizarin red (FIG. 27C) and expression of osteocyte markers (FIG. 27D). Furthermore, therapeutically reprogrammed pluripotent human stem cells were differentiated into hepatocytes according to the teachings of the present invention by culturing in hMASC medium containing hepatocyte growth factor (HGF) and FGFA as evidenced by morphology (FIG. 27E) and expression of hepatocyte-endodermal gene markers (FIG. 27F).

FIG. 28 depicts the karyotype of a human PGS cell line made according to the teachings of the present invention.

### DEFINITION OF TERMS

The following definition of terms is provided as a helpful reference for the reader. The terms used in this patent have specific meanings as they related to the present invention. Every effort has been made to use terms according to their ordinary and common meaning. However, where a discrepancy exists between the common ordinary meaning and the following definitions, these definitions supercede common usage.

Committed: As used herein, “committed” refers to cells which are considered to be permanently committed to a specific function. Committed cells are also referred to as “terminally differentiated cells.”

Dedifferentiation: As used herein, “dedifferentiation” refers to loss of specialization in form or function. In cells, dedifferentiation leads to an a less committed cell.

Differentiation: As used herein, “differentiation” refers to the adaptation of cells for a particular form or function. In cells, differentiation leads to a more committed cell.

Embryo: As used herein, “embryo” refers to an animal in the early stages of growth and differentiation that are characterized implantation and gastrulation, where the three germ layers are defined and established and by differentiation of the germs layers into the respective organs and organ systems. The three germ layers are the endoderm, ectoderm and mesoderm.

Embryonic Stem Cell: As used herein, “embryonic stem cell” refers to any cell that is totipotent and derived from a developing embryo that has reached the developmental stage to have attached to the uterine wall. In this context embryonic stem cell and pre-embryonic stem cell are equivalent terms. Embryonic stem cell-like (ESC-like) cells are totipotent or pluripotent cells not directly isolated from an embryo. ESC-like cells can be derived from primordial sex cells that have been dedifferentiated in accordance with the teachings of the present invention.

Fetal Stem Cell: As used herein, “fetal stem cell” refers to a cell that is multipotent and derived from a developing multi-cellular fetus that is no longer in early or mid-stage organogenesis.

Germ Cell: As used herein, “germ cell” refers to a reproductive cell such as a spermatocyte or an oocyte, or a cell that will develop into a reproductive cell.

Maturation: As used herein, “maturation” refers to a process of coordinated steps either forward or backward in the differentiation pathway and can refer to both differentiation or de-differentiation. As used herein, maturation is synonymous with the terms develop or development when applied to the process described herein.

Multipotent: As used herein, “multipotent” refers to cells that can give rise to several other cell types, but those cell types are limited in number. An example of a multipotent cells is hematopoietic cells—blood stem cells that can develop into several types of blood cells but cannot develop into brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells.

Pluripotent: As used herein, “pluripotent” refers to cells that can give rise to any cell type except the cells of the placenta or other supporting cells of the uterus.

Pluripotent Germ Stem Cell: As used herein “pluripotent germ stem cell” or “PGS” refers to a primordial sex cell that has been therapeutically reprogrammed to be pluripotent and can be maintained in culture.

Post-natal Stem Cell: As used herein, “post-natal stem cell” refers to any cell that is multipotent and derived from a multi-cellular organism after birth.

Pre-embryo: As used herein, “pre-embryo” refers to a fertilized egg in the early stage of development prior to cell division. During the pre-embryonic stage the initial stages of cleavage are occurring.

Pre-embryonic Stem Cell: See “Embryonic Stem Cell” above.

Primordial Sex Cell: As used herein, “primordial sex cell” refers to any diploid cell that is derived from the male or female mature or developing gonad, is able to generate cells that propagate a species and contains a diploid genomic state. Primordial sex cells can be-quiescent or actively dividing. These cells include male gonocytes, female gonocytes, spermatogonial stem cells, ovarian stem cells, oogonia, type-A spermatogonia, Type-B spermatogonia. Primordial sex cells are also known as germ-line stem cells (GSC).

Primordial Germ Cell: As used herein, “primordial germ cell” refers to cells present in early embryogenesis that are destined to become germ cells.

Reprogramming: As used herein “reprogramming” refers to the resetting of the genetic program of a cell such that the cell exhibits pluripotency and has the potential to produce a fully developed organism.

Responsive: As used herein, “responsive” refers to the condition of a cell, or group of cells, wherein they are susceptible to and can function accordingly within a cellular environment. Responsive cells are capable of responding to and functioning in a particular cellular environment, tissue, organ and/or organ system.

Somatic Stem Cells: As used herein, “somatic stem cells” refers to diploid multipotent or pluripotent stem cells. Somatic stem cells are not totipotent stem cells.

Therapeutic Cloning: As used herein, “therapeutic cloning” refers to the cloning of cells using nuclear transfer methods including replacing the nucleus of an ovum with the nucleus of another cell and stem cells derived from the inner cell mass.

Therapeutic Reprogramming: As used herein, “therapeutic reprogramming” refers to the process of maturation wherein a stem cell is exposed to stimulatory factors according to the teachings of the present invention to yield either pluripotent, multipotent or tissue-specific committed cells. Therapeutically reprogrammed cells are useful for implantation into a host to replace or repair diseased, damaged, defective or genetically impaired tissue. The therapeutically reprogrammed cells of the present invention do not possess non-human sialic acid residues.

Totipotent: As used herein, “totipotent” refers to cells that contain all the genetic information needed to create all the cells of the body plus the placenta. Human cells have the capacity to be totipotent only during the first few divisions of a fertilized egg.

### DETAILED DESCRIPTION OF THE INVENTION

The present invention provides biologically useful pluripotent therapeutically reprogrammed cells, generated from post-natal stem cells, having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications.

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into. pluripotent types (Kanatsu-Shinohara M. et al. Generation of pluripotent stem cells from neonatal mouse testis. Cell 119:1001-12, 2004).

The ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct-4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G₀/G₁ phase of the cell cycle. In mice and rats these gonocytes resume division within a few days after birth to generate spermatogonial stem cells and eventually undergo differentiation and meiosis related to spermatogenesis.

Primordial sex cells are directly responsible for generating the cells required for fertilization and eventually a new round of embryogenesis to create a new organism. Primordial sex cells are not programmed to die and are of a quality comparable to that of an embryonic state.

Embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage.

For the purpose of this discussion, an embryo and a fetus are distinguished based on the developmental stage in relation to organogenesis. The pre-embryonic stage refers to a period in which the pre-embryo is undergoing the initial stages of cleavage. Early embryogenesis is marked by implantation and gastrulation, wherein the three germ layers are defined and established. Late embryogenesis is defined by the differentiation of the germ layer derivatives into formation of respective organs and organ systems. The transition of embryo to fetus is defined by the development of most major organs and organ systems, followed by rapid fetal growth.

Embryogenesis is the developmental process wherein an oocyte fertilized by a sperm begins to divide and undergoes the first round of embryogenesis where cleavage and blastulation occur. During the second round, implantation, gastrulation and early organogenesis takes place. The third round is characterized by organogenesis and the last round of embryogenesis, wherein the embryo is no longer termed an embryo, but a fetus, is when fetal growth and development occurs.

During embryogenesis the first two tissue lineages arising from the morulae post-cleavage and compaction are the trophectoderm and the primitive endoderm, which make major contributions to the placenta and the extraembryonic yolk sac. Shortly after compaction and prior to implanting the epiblast or primitive ectoderm begins to develop.

The epiblast provides the cells that give rise to the embryo proper. Blastulation is complete upon the development of the epiblast stem cell niche wherein pluripotent cells are housed and directed to perform various developmental tasks during development, at which time the embryo emerges from the zona pellucida and implants to the uterine wall.

Implantation is followed by gastrulation and early organogenesis. By the end of the first round of organogenesis, all three germ layers will have been formed; ectoderm, mesoderm and definitive endoderm and basic body plan and organ primordia are established. Following early organogenesis, embryogenesis is marked by extensive organ development at which time completion marks the transformation of the developing embryo into a developing fetus which is characterized by fetal growth and a final round of organ development. Once embryogenesis is complete, the gestation period is ended by birth, at which time the organism has all the required organs, tissues and cellular niches to function normally and survive post-natally.

The process of embryogenesis is used to describe the global process of embryo development as it occurs, but on a cellular level embryogenesis can be described and/or demonstrated by cell maturation.

Fetal stem cells have been isolated from the fetal bone marrow (hematopoietic stem cells), fetal brain (neural stem cells) and amniotic fluid (pluripotent amniotic stem cells). In addition, stem cells have been described in both adult male and fetal tissues. Fetal stem cells serve multiple roles during the process of organogenesis and fetal development, and ultimately become part of the somatic stem cell reserve.

Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory.

During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress.

There is a history of cellular therapy for the treatment of a variety of diseases but the majority of the use has been in bone marrow transplantation for hematopoietic disorders, including malignancies. In bone marrow transplantation, an individual's immune system is restored with the transplanted bone marrow from another individual. This restoration has long been attributed to the action of hematopoietic stem cells in the bone marrow.

There is increasing evidence that stem cells can be differentiated into particular cell types in vitro and shown to have the potential to be multipotent by engrafting into various tissues and transit across germ layers and as such have been the subject of much research for cellular therapy. As with conventional types of transplants, immune rejection is the limiting factor for cellular therapy. The recipient individual's phenotype and the phenotype of the donor will determine if a cell or organ transplant will be tolerated or rejected by the immune system.

Therefore, the present invention provides methods and compositions for providing functional immunocompatible stem cells for cellular regenerative/reparative therapy.

In an embodiment of the present invention, methods and compositions for therapeutically reprogramming post-natal stem cells are provided. Therapeutic reprogramming refers to a maturation process wherein a stem cell is exposed to stimulatory factors according the teachings of the present invention to yield pluripotent, multipotent or tissue-specific committed cells. The process of therapeutic reprogramming can be performed with a variety of stem cells including, but not limited to, therapeutically cloned cells, hybrid stem cells, embryonic stem cells, fetal stem cells, multipotent post-natal stem cells (adult progenitor cells), adipose-derived stem cells (ADSC) and primordial sex cells.

The therapeutic reprogramming method of the present invention is suitable for reprogramming cells from a variety of animals including, but not limited to, primates, rodents, sheep, cattle, goats, pigs, horses, etc. In one embodiment, the primate is a human.

Therapeutic reprogramming takes advantage of the fact that certain stem cells are relatively easily to obtain, such as spermatogonial stem cells and adipose-derived stem cells, and epigenetically reprograms these cells by exposure to stimulatory factors. These therapeutically reprogrammed cells have changed their maturation state to either a more committed cell lineage or a less committed cell lineage. Therapeutically reprogrammed cells are therefore capable of repairing or regenerating disease, damaged, defective or genetically impaired tissues.

Therapeutic reprogramming uses stimulatory factors, including without limitation, chemicals, biochemicals and cellular extracts to change the epigenetic programming of cells. These stimulatory factors induce, among other results, genomic methylation and/or acetylation changes in the donor DNA.

In one specific embodiment of the present invention, primordial sex cells (PSC) are therapeutically reprogrammed. Primordial sex cells, residing in the lining of the seminiferous tubules of the testes and the lining of the ovaries (the spermatogonia and oogonia, respectively) have been determined to possess diploid (2N) genomes remarkably undamaged by to the effects of aging and cell division. Thus, PSCs possess genomes in a nearly physiologically prime state. A non-limiting example of a PSC particularly useful in an embodiment of the present invention is a spermatogonial stem cell. According to the teachings herein, therapeutically reprogrammed PSC cells are prepared for the maturation process using means similar to that experienced by stem cells present in the developing embryo and fetus during embryogenesis and organogenesis.

In an embodiment of the present invention, spermatogonial stem cells are therapeutically reprogrammed by culture in the presence of cell growth promoting and maintenance and maturation factors in PM-1 medium in the absence of feeder cells (Example 3). PM-1 medium contains the signals necessary for spermatogonial stem cells to be therapeutically reprogrammed into pluripotent embryonic stem cell-like cells. The cell growth and maturation factors useful for the therapeutic reprogramming of PSCs using PM-1 media in a low serum environment include, but are not limited to, epidermal growth factor (EGF), fibroblast growth factor 2 (FGF2), glial cell derived neurotrophic factor (GDNF) and leukemia inhibitory factor (LIF). Additionally, an Expansion Medium containing growth factors and other cell growth promoting and maintenance factors is disclosed in Example 3 (Table 4). Expansion medium is suitable for culturing therapeutically reprogrammed pluripotent embryonic stem cell-like cells.

In an embodiment of the present invention, spermatogonial stem cells, isolated from human males undergoing standard castration surgical procedures and who have been on long-term hormone (estrogen) treatment, are therapeutically reprogrammed by culture in the presence of cell growth promoting and maintenance and maturation factors in PM-10, PM-20 or PM-100 medium in the absence of feeder cells and serum (Example 8). PM-10 and PM-20 media contains the signals necessary for human spermatogonial stem cells to be therapeutically reprogrammed into pluripotent embryonic stem cell-like cells. The cell growth and maturation factors useful for the therapeutic reprogramming of PSCs using PM-10 and PM-20 or PM-100 media in a serum-free environment include, but are not limited to, epidermal growth factor (EGF), fibroblast growth factor 2 (FGF2), glial cell derived neurotrophic factor (GDNF) and leukemia inhibitory factor (LIF).

Embodiments of the present invention provide methods for further maturing or differentiating therapeutically reprogrammed human cells, stem cells and primordial sex cells into more committed cell lineages in a post-natal environment to provide more committed cells for use in cellular regenerative/reparative therapy. In addition the maturation and differentiation process provides therapeutic cells that can be used to treat or replace damaged cells in pre- and post-natal organs.

In one embodiment of the present invention, PM-3 medium has been used to differentiate therapeutically reprogrammed PSCs into beating cardiac myocytes. It is within the scope of the present invention to differentiate therapeutically reprogrammed PSCs with PM-3 medium into other cell lineages including, but not limited to, neural cells, hematopoietic cells, bone cells, and others. The cell growth and maturation factors useful for maturation and differentiation of PSCs and therapeutically reprogrammed PSCs using PM-3 media include, but are not limited to, epidermal growth factor (EGF), fibroblast growth factor 2 (FGF2), glial cell derived neurotrophic factor (GDNF) and leukemia inhibitory factor (LIF).

In another embodiment, PM-1-AZA medium has been used to differentiate therapeutically reprogrammed human PSCs into beating cardiac myocytes. It is within the scope of the present invention to differentiate therapeutically reprogrammed human PSCs into other cell lineages including, but not limited to, neural cells, hematopoietic cells, bone cells, and others. The cell growth and maturation factors useful for therapeutic reprogramming and maturation and differentiation of human PSCs and therapeutically reprogrammed human PSCs include, but are not limited to, EGF, FGF-2, GDNF, LIF, platelet-derived growth factor BB (PDGF-BB), transforming growth factor-β3 (TGF-β3), fibroblast growth factor 8 (FGF-8) and sonic hedgehog (SHH).

The therapeutically reprogrammed cells produced from primordial sex cells have been termed pluripotent germ-line stem (PGS) cells. These PGS cells made according to the teachings of the present invention demonstrate that spermatogonial stem cells (SSCs) isolated from post-natal mouse testes retain the plasticity for reprogramming and become pluripotent stem cells, as they contributed to normal embryo development after blastocyst injection. They can also be directed to differentiate into different cell lineages in vitro.

First, regarding which subpopulation(s) among SSCs in the post-natal mouse testis contributes to PGS cell formation, the c-kit positive cell population is preferred for reprogramming into PGS cells. The c-kit negative cells, while also exhibiting pluripotent markers and high telomerase activity, are committed to the gonadal lineage. Surprisingly, neither the c-kit positive nor the c-kit negative cells induced teratoma formation after transplantation into immune compromised mice. The non-teratogenicity of PGS cells is unexpected, since embryonic stem (ES) cell-like multipotent stem cells form teratomas. Because germ-line stem cells have been found to be non-teratogenic after 13.5 dpc (days post conception) following imprinting, the PGS cells are different from reported ES cell-like cells in terms of developmental stages and potentiality. The reprogrammed PGS cells reach an early developmental stage that is pluripotent but not at the level of ES cells. Therefore, PGS cells are a superior cell source for cell replacement therapy that is devoid of the limitations, but retain the advantages, of ES and adult stem cells.

Imprinting is an important feature of cells for cell replacement therapy because it preserves or eliminates genetic traits of the donor cells. In male germ cells, genomic imprinting is erased during the fetal stage, and male-specific imprinting begins to be acquired around birth in prospermatogonia; the process is completed after birth. The molecular events involved in the imprinting process are believed to resemble those occurred during reprogramming. It has been shown that the conversion of primordial germ cells (PGC). cells to embryonic germ cells is dependent on polypeptide growth factors including stem cell factor, LIF and bFGF. Growth factors such as bFGF, LIF, GDNF and EGF are important factors to convert SSC into pluripotent germ cells. This process, revealed by using a reporter gene tracking system, involves a rapid down regulation of Oct-4 after initiation of culture followed by up-regulation of Oct-4 during formation of PGS colonies.

PGS cells have lower expression of SSEA-1 compared to ES cells and ES-like cells. SSEA-1 is a member of the carbohydrate stage specific embryonal antigen family and has been shown to be involved in tumor invasion and metastasis. Selection and enrichment of human therapeutic PGS cells by SSEA-1, for example, may improve the quality of transplantable PGS population. The derivation and up-scale production of non-teratogenic PGS cells isolated from post-natal and adult human testes, capable of differentiating into functional phenotypes in vivo, will have immediate impact in stem cell biology and application.

The therapeutically reprogrammed human cells made in accordance with the teachings of the present invention are useful in a wide range of therapeutic applications for cellular regenerative/reparative therapy. For example, and not intended as a limitation, the therapeutically reprogrammed human cells of the present invention can be used to replenish stem cells in mammals whose natural stem cells have been depleted due to age or ablation therapy such as cancer radiotherapy and chemotherapy. In another non-limiting example, the therapeutically reprogrammed human cells of the present invention are useful in organ regeneration and tissue repair. In one embodiment of the present invention, therapeutically reprogrammed human cells can be used to reinvigorate damaged muscle tissue including dystrophic muscles and muscles damaged by ischemic events such as myocardial infarcts. In another embodiment of the present invention, the therapeutically reprogrammed human cells disclosed herein can be used to ameliorate scarring in animals, including humans, following a traumatic injury or surgery. In this embodiment, the therapeutically reprogrammed human cells of the present invention are administered systemically, such as intravenously, and migrate to the site of the freshly traumatized tissue recruited by circulating cytokines secreted by the damaged cells. In another embodiment of the present invention, the therapeutically reprogrammed human cells can be administered locally to a treatment site in need or repair or regeneration.

Stem cells are not universally susceptible to the maturation process of the present invention. Therefore the present inventors have developed a therapeutic reprogramming process whereby post-natal human stem cells are induced into a state whereby they are susceptible to maturation factors. This therapeutic reprogramming process can be accomplished by incubation with stimulatory factors under suitable conditions and for a time sufficient to render the donor cell susceptible for maturation.

The following examples are meant to illustrate one or more embodiments of the invention and are not meant to limit the invention to that which is described below.

### EXAMPLES

### Example 1

### Isolation of Primordial Sex Cells from Murine Testes

Testes can be isolated from pre-natal, embryonic, post-natal and adult testes using the following methods and additional methods known to persons of ordinary skill in the art.

Male germ stem cells were isolated from 0- to 3-day old OG2 male gonads. Transgenic OG2 mice express green fluorescent protein (GFP). Approximately thirty pups were used in each trial (totally 25 trials). After mincing, testes were digested in DPBS containing collagenase (lmg/ml), DNAse-1 (1 μg/ml) and EDTA (5 mM). Testicular cell culture was performed according to methods well known to persons skilled in the art. In brief, cells were allocated in gelatin-coated (0.1%) culture dishes. The next day, floating cells were collected and passed to a secondary culture plate (1×10⁵ cells per 1.2 cm²) in PM-1 medium containing mouse EGF (R&D Systems, Minneapolis), human bFGF (R&D), ESGRO (murine leukemia inhibitory factor, Chemicon) and recombinant GDNF (R&D). After 2-4 weeks in culture, the GFP positive colonies were mechanically transferred to mitomycin C-treated murine embryonic fibroblast (MEF) feeders. After propagation 3-4 times with mechanical transfer to new MEF, the colonies were established and could be removed from the culture plate enzymatically for further expansion and storage. Testicular cells were also isolated from neonatal, non-transgenic ICR mice and cultured as in neonatal OG2 mouse testicular stem cells. Additionally, testicular cells were isolated from adult OG2 mice 4- to 6-week old (n=4) whose testes were surgically operated and secured in the abdominal wall to become cryptorchid in 2-3 months with arrested spermatogenesis 30. In addition, non-cryptorchid adult OG2 mice used for cell isolation. Testicular cells isolated from the adult mice were also sorted for GFP before they were cultured on MEF feeders in PM-1 medium as described above.

Mouse embryonic fibroblast (MEF) feeders were prepared using standard procedures with 12.5 dpc CD-1 mouse embryos. The embryos were eviscerated before trypsinization, and the dissociated cells were plated onto 150-mm plates with plating density at ˜1.5 embryos per plate. After the initial plating, MEFs were split 1:5 and then frozen (passage 1). Thawed MEFs (P1) were passed only once for expansion purposes prior to Mitomycin C treatment. MEF feeders were plated in a density of 50-60×10³ per cm². Fresh MEF feeders were used for PGS cell derivation every 7-10 days.

### Example 2

### Isolation of Primordial Sex Cells from Ovaries

The animal is anesthetized and the ovaries are removed. Alternatively, primordial sex cells (PSCs) can be isolated from a punch biopsy the ovaries. The PSCs are then isolated with the assistance of a microscope. Primordial sex cells have stem cell morphology (i.e. large, round and smooth) and are mechanically retrieved from the ovaries.

### Example 3

### Therapeutic Reprogramming of Germ-Line Stem Cells into Therapeutic Cells

Primordial sex cells were isolated as described in Example 1. The cells were then plated out at a density of 200,000 cells/3.8 cm² in PM-1 medium and then incubated overnight at 37° C. for differential adhesion which separates somatic cells (attached) from germ line stem cells, spermatogonial stem cells and/or primordial sex cells (suspended). This day was designated Day 0.

StemPro®-34 Complete Medium (Invitrogen Corporation, Carlsbad, Calif.) is comprised of StemPro®-34 Serum Free Medium (SFM) and StemPro®-34 Nutrient Supplement and disclosed in U.S. Pat. No. 6,733,746 B2, the contents of which are herein incorporated by reference in their entirety, particularly Tables 1, 2 and 3 in columns 17-18. For the purposes of this disclosure, StemPro®-34 Complete Medium will be referred to as Stem Cell Basal Medium. Tables 2 and 3, which list the components of the StemPro-34 Complete Medium used in the cell culture medium of the present invention are reproduced below exactly as they are found in the '746 patent.

TABLE 1
PM-1 Medium

StemPro ®-34 Complete Medium
1% FBS (Hyclone)
2 mM L-glutamine (Invitrogen 35050-061)
Minimal Essential Medium (MEM) vitamin solution (Invitrogen)
0.1 mM MEM non-essential amino acids solution (Invitrogen
11140-050)
10⁻⁴ ascorbic acid
10 μg/mL d-biotin
80 ng/mL β-estradiol
60 ng/mL progesterone
5 mg/mL bovine albumin
1 μL/mL DL-lactic acid
30 μg/mL pyruvic acid
6 mg/mL D-(+)-glucose
30 nM sodium selenite
60 μM putrescine
100 μg/mL transferrin
25 μg/mL insulin
1X penicillin/streptomycin (Invitrogen 15070-063)
5 × 10⁻⁵ M β-mercaptoethanol (Sigma M7522)
20 ng/mL recombinant human epidermal growth factor (rhEGF)
10 ng/mL recombinant human fibroblast growth factor 2 (rhFGF2)
2 ng/mL recombinant human glial cell derived neurotrophic factor
(rhGDNF)
1,000 units/mL ESGRO ® (mouse leukemia inhibitory factor, LIF)
(Chemicon)

TABLE 2
StemPro ™-34 Nutrient Supplement Formulation (40X)

| | Concentration (mg/L) |
|---|---|
| Ingredient | (About) |
| Human Serum Albumin | 200,000 |
| HUMAN EX-CYTE ® | 200 |
| Ethanolamine - HCl | 400 |
| Sodium Selenite | 0.2 |
| Hydrocortisone | 2 |
| D,L-Tocopherol | 0.8 |
| Iron Saturated Human Transferrin | 4 |
| Human Zinc Insulin | 400 |
| N-acetyl-L-cysteine | 7 |
| 2-Mercaptoethanol | 180 |

TABLE 3
StemPro ™-34 Medium Formulation

| | | |
|---|---|---|
| | Concentration Range | A Preferred |
| | in 1X Medium | Embodiment |
| | (mg/mL) | (mg/L) |
| Ingredient | (About) | (About) |

Inorganic Salts

| | | |
|---|---|---|
| CaCl₂ | 1-500 | 165 |
| KCL | 1-500 | 330 |
| KNO₃ | 0.008-0.8 | 0.08 |
| MgSO₄ | 10-500 | 100 |
| NaCl | 3000-9000 | 4500 |
| NaHCO₃ | 100-4000 | 3000 |
| NaH₂PO₄.water | 10-750 | 125 |

Amino Acids

| | | |
|---|---|---|
| L-Alanine | 5-250 | 25 |
| L-Asparagine (free base) | 5-150 | 25 |
| L-Arginine HCl | 10-250 | 84 |
| L-Aspartic Acid | 5-125 | 30 |
| L-Cysteine 2.HCl | 1-200 | 90 |
| L-Glutamic Acid | 5-500 | 75 |
| Glycine | 5-200 | 30 |
| L-Histidine.HCl.water | 5-200 | 42 |
| L-Isoleucine | 5-500 | 105 |
| L-Leucine | 25-500 | 105 |
| L-Methionine | 5-500 | 30 |
| L-Phenylalanine | 5-500 | 70 |
| L-Proline | 5-500 | 40 |
| L-Serine | 5-500 | 40 |
| L-Threonine | 5-500 | 100 |
| L-Lysine.HCl | 25-500 | 150 |
| L-Tryptophan | 2-100 | 15 |
| L-Tyrosine (disodium salt) | 25-500 | 100 |
| L-Valine | 5-500 | 95 |

Vitamins

| | | |
|---|---|---|
| Biotin | 0.01-1.0 | 0.01 |
| D-Ca Pantothenate | 0.05-10.0 | 4 |
| Choline Chloride | 1-150 | 4 |
| Folio Acid | 0.1-10.0 | 4.00 |
| i-Inositol | 1-75 | 7 |
| Niacinamide | 0.1-10.0 | 4 |
| Pyridoxal.HCl | 0.1-10.0 | 4 |
| Riboflavin | 0.01-2.0 | 0.4 |
| Thiamine.HCl | 0.1-10.0 | 4 |
| Vitamin B₁₂ | 0.001-5.0 | 0.001 |

Other Components

| | | |
|---|---|---|
| Human Serum Albumin | 1000-15,000 | 5000 |
| NaSeO₃ | 0.001-0.01 | 0.02 |
| D-Glucose | 2000-9000 | 4500 |
| Phenol Red | 0.5-30 | 15 |
| HEPES | 1000-7000 | 6000 |
| Sodium Pyruvate | 10-300 | 110 |
| HUMAN EX-CYTE ® | 1-15 | 5 |
| Ethanolamine | 1-25 | 10 |
| Hydrocortisone | 0.003-0.07 | 0.04 |
| D,L-Tocopherol | 0.005-0.05 | 0.02 |
| Iron Saturated Human Transferrin | 10-500 | 100 |
| Human Zinc Insulin | 1-25 | 10 |
| N-acetyl-L-cysteine | 16-660 | 160 |
| 2-Mercaptoethanol | 2-8 | 4 |

PM-1 medium is specifically designed for the culture and maintenance of primordial sex cells (PSC) and their initial reprogramming into pluripotent germ-line stem (PGS) cells. The PM-1 media described above is an exemplary embodiment of the therapeutic reprogramming method of the present invention. It will be understood by persons skilled in the art that the concentrations of the components of PM-1 can vary and still achieve the intended results. In an embodiment of the present invention, the concentration of rhEGF can range from approximately 10 ng/mL to approximately 40 ng/mL, the concentration of rhFGF2 can range from approximately 10 ng/mL to approximately 40 ng/mL, the concentration of rhGDNF can range from approximately 2 ng/mL to approximately 40 ng/mL and the concentration of ESGRO® mouse LIF can range from approximately 1,000 units/mL to approximately 10,000 units/mL.

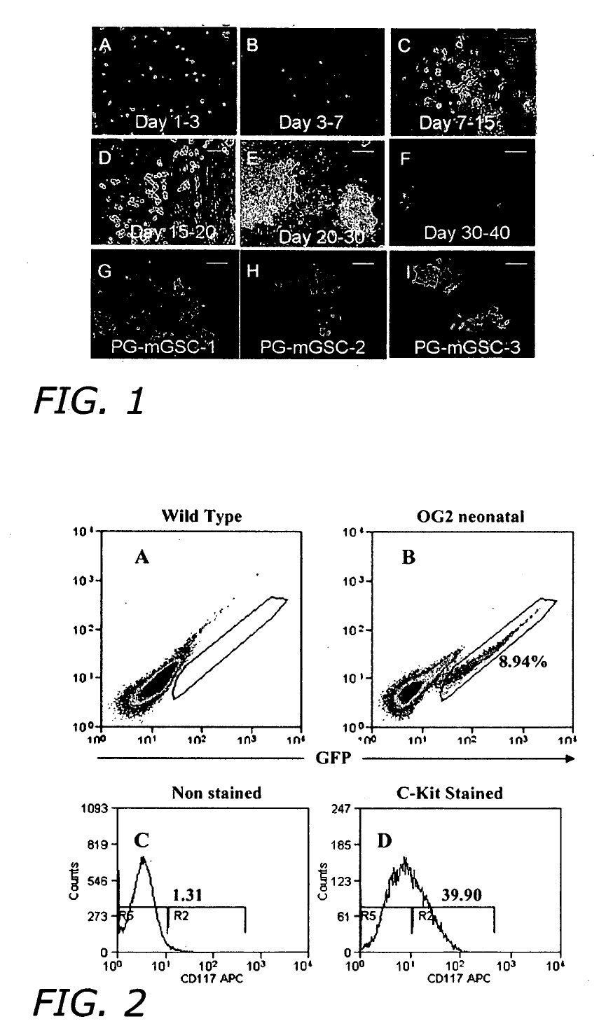

The following day (Day 1), the floating cells were collected and plated onto 0.1% gelatinized plates and cultured in PM-1. At this stage the cells were 8-10 micron in size, round with appearance of pseudopods and non-adherent (FIG. 1A). Cells cultured in PM-1 demonstrated morphology changes between days 3 and 15 (day 3-7 cells are depicted in FIG. 1B and day 7-15 cells in FIG. 1C. During this phase of therapeutic reprogramming the cell divide in distinct colonies and are approximately 10-15 micron in size, and is adherent. Colony size was less than 50 total cells.

In the next phase of therapeutic reprogramming, beginning at approximately day 17 (FIG. 1D) the colony size increases to 50-200 cells and cells grow in close contact with each other. Cellular morphology is also different in this phase of therapeutic reprogramming. The cells appear to lose their roundness and only cells 10 micron or smaller survive.

At approximately day 26 (FIG. 1E) the colony size grows to greater than 200 cells. The cells at this point are approximately 10 micron in size, and expressed high levels of the pluripotent stem cell marker Oct-4 (FIG. 1F).

The therapeutically reprogrammed cells were then picked and plated onto dishes containing inactivated primary mouse embryonic fibroblasts. At this point the therapeutic reprogramming is completed and the reprogrammed cells can be expanded in expansion medium (Table 4) or continued to be cultured in PM-1 (Table 1), PM-10, PM20, or PM-101 and differentiated.

TABLE 4
Expansion Medium

Dulbecco's Modified Eagle's Medium (D-MEM, Specialty Media
SLM-220B)
15% FBS (Hyctone)
2 mM L-glutamine (Invitrogen 35050-061)
0.1 mM MEM non-essential amino acids (Invitrogen 11140-050)
1X penicillin/streptomycin (Invitrogen 15070-063)
0.1 mM beta-mercaptoethanol (Sigma M7522)
1,000 units/mL ESGRO ® (mouse LIF)

Expansion medium is specifically designed for the culture and maintenance of pluripotent therapeutically reprogrammed cells. The expansion medium described above is an exemplary embodiment of the therapeutic reprogramming method of the present invention. It will be understood by persons skilled in the art that the concentrations of the components of expansion medium can vary and still achieve the intended results. In an embodiment of the present invention, the concentration of ESGRO® mouse LIF can range from approximately 1,000 units/mL to approximately 10,000 units/mL.

### Example 4

### Evaluation of Murine Pluripotent Cell Lines by Therapeutic Reprogramming

A reporter gene inserted into the distal regulatory element of the Oct-4 genomic fragment was used to visualize pluripotent germ line cell development in the mouse. Transgenic OG2 mice which express green fluorescent protein (GFP) were used for isolation and reprogramming of germ-line stem cells. Isolated testes cells were cultured on gelatin-coated dishes in PM-1 medium (Example 3) containing FBS (Hyclone) and growth factors including GDNF, bFGF, LIF and EGF for reprogramming. Shortly after culture, the GFP-positive signals diminished significantly and disappeared after a few days (FIG. 1A-B). Thereafter, cells exhibited distinct morphological changes that include chain-like structures and colonies (FIGS. 1C-E).

Appearance of GFP-positive cells within colonies after 3-4 weeks of culture (FIG. 1F) suggesting a reprogramming process has occurred. As soon as GFP-positive colonies were formed, they were transferred to mouse embryonic fibroblast (MEF) feeder and cultured in the expansion medium (Example 3). This protocol for pluripotent germ line stem cell (PGS) formation was reproducible, with derivation efficiency of ˜30% (3 cell lines derived from 10 trials; (FIG. 1G-I). The PGS colony formation can be advanced by approximately two weeks when GFP-positive single cells were sorted by FACS immediately after isolation and differential adhesion, and were directly cultured on MEF feeder cells. The Oct-4-GFP signals disappeared within days but reappeared in 2 weeks.

The following antibodies and methods were used in the characterization of the PGS cells. The following primary antibodies were acquired from Chemicon, Temecula, Calif.: mouse anti-SSEA-1, goat anti-mouse cardiac troponin-1, mouse anti-myosin heavy chain, rabbit anti-glial fibrillary acidic protein (GFAP), mouse anti-neurofilament, and mouse anti-nestin. Other antibodies used are: mouse anti-nanog (bethyl, Montgomery, Tex.), APC conjugated anti-c-kit (BD Biosciences, San Jose, Calif.), goat anti-mouse IgM-FITC (BD Biosciences, San Jose, Calif.), Alexa Fluor 488-conjugated goat anti-mouse IgG (Molecular Probes, Carlsbad, Calif.) and goat anti-mouse Texas red (Abcam, Cambridge, Mass.). Alkaline phosphatase staining was carried out using the StemTAG kit (Cell Biolabs, San Diego, Calif.) according to manufacturer's protocol. Cultured cells were fixed in 2% paraformaldehyde buffered in PBS for 30 minutes at room temperature and stored in PBS at 4° C. For immunocytochemistry, cells were permeablized with 1× Cytoperm (BD Biosciences, San Jose, Calif.) for 15 minutes and subsequently incubated in 2% (w/v) bovine serum albumin (Sigma, St Louis, Mo.), 2% (v/v) normal goat serum/1× Cytoperm-PBS for 30 minutes both at room temperature. Primary antibodies were diluted in 2% BSA 2% GS/lx Cytoperm-PBS and incubated for 1-3 hours at 4° C. After two washes, fluorescent secondary antibody was diluted in 2% BSA 2% GS/1× Cytoperm-PBS and incubated for 1 hour at 4° C. in the dark. Cells were washed twice, wrapped in foil and stored at 4° C. until microscopic analysis.

Cells were sorted on the Influx Cell Sorter (Cytopeia, Seattle, Wash.). Cells containing the Oct-4-GFP construct were stained with CD117 APC (BD Pharmingen, San Diego, Calif.). GFP excitation was attained with 488nm solid state Coherent laser (Coherent, Santa Clara, Calif.) and emission light collected through a 530-40 dichroic mirror. APC excitation was attained with 638nm solid state Coherent laser and emission light collected through a 710-40 dichroic mirror.

Among many factors involved in germ line development, c-kit, the receptor of stem cell factor, plays a critical role in the fate of germ cell development. In the mouse embryo, c-kit is highly expressed in primordial germ cells (PGC) and its expression is down regulated when PGC differentiate to gonocytes and spermatogonial stem cells. Therefore the Oct-4-GFP positive PGS cells were sorted (FIG. 2A-D) on the basis of c-kit expression and both c-kit positive and c-kit negative cells were directly cultured on MEF feeder cells.

For some experiments, fresh PGS colonies were dissociated and cells were stained with anti-SSEA-1 antibody following by goat-anti rnouse IgM conjugated with PE-Cy7 (BD, Biosciences, San Jose, Calif.).

Three PGS cell lines were generated from different sources of PSCs: PG-mGSC-1 (from neonatal mouse PSC) PG-mGSC-2 and PG-mGSC-3 (from adult mouse PSC). PGS cell lines have also been generated from adult OG2 and wild type ICR mice to demonstrate the reproducibility of the therapeutic reprogramming methods for both adult and non-transgenic animals. PGS colonies were generated only from c-kit positive cells (4/4) but not c-kit negative cells (0/4).

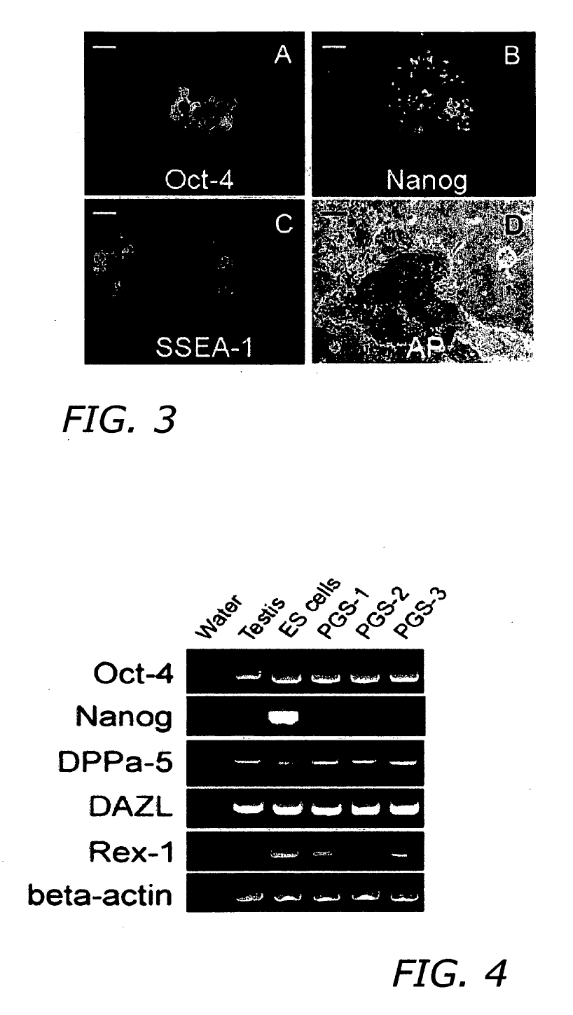

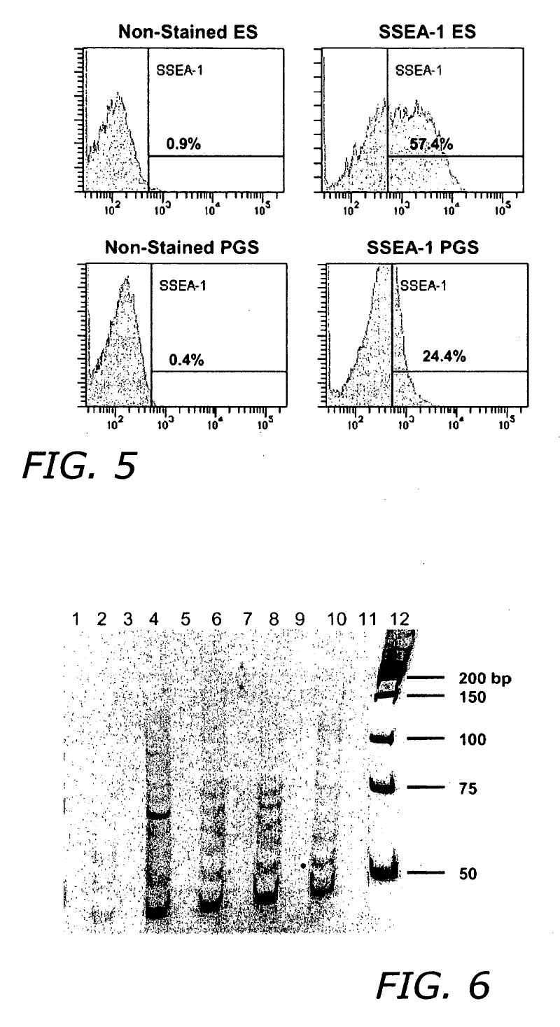

Characterization of the PGS cell lines for pluripotent markers other than Oct-4 (GFP+) showed that they co-expressed with SSEA-1 and Nanog (ES cell-specific), as well as alkaline phosphatase (FIG. 3). Moreover, RT-PCR analysis confirmed that PGS cells also expressed Rex-1 and Dppa5, two additional pluripotent markers in addition to Oct-4 and Nanog. All PGS cells expressed DAZL, a germ cell specific marker (FIG. 4). Analysis by flow cytometry showed that about 20% of the PGS cells had low SSEA-1 expression (FIG. 5). In contrast, about 60% of ES cells examined expressed SSEA-1 (FIG. 5).

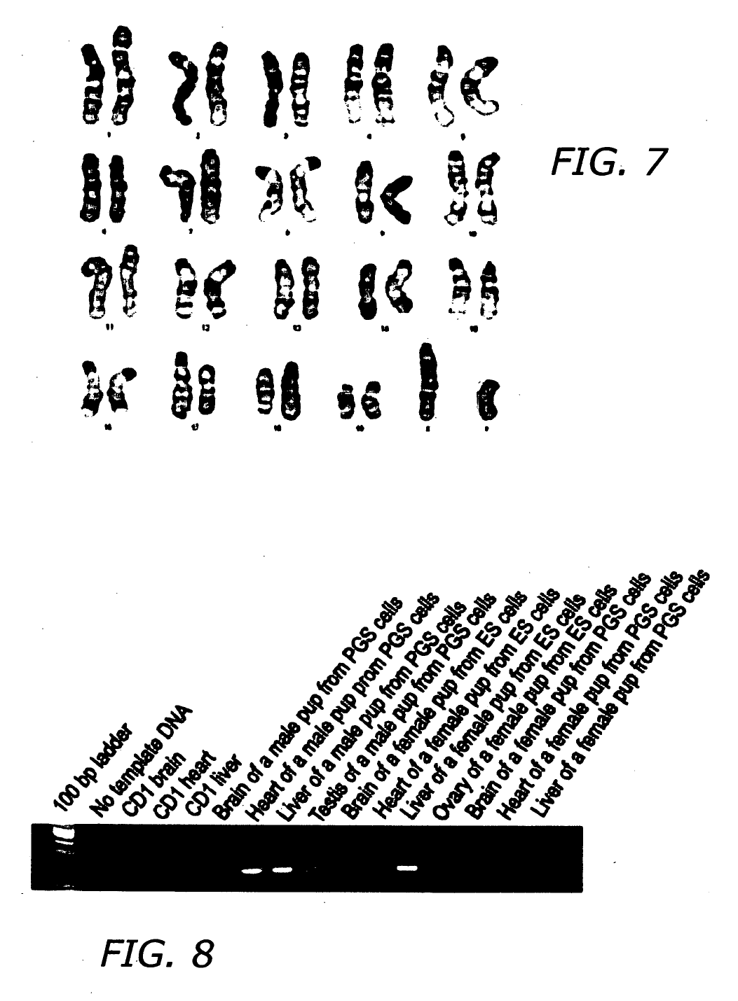

In addition, PGS cell lines were evaluated for telomerase activity and karyotyped. For determination of telomerase activity, cell extracts were isolated from PGS cell lines (passage 10 and higher), freshly isolated Oct-4+/ckit+ sorted cells and Oct-4+/ckit− sorted cells using CHAPS lysis buffer containing 150U/mL RNase. Cell lysates were centrifuged for 20 minutes at 12,000×g, 4° C. and the supernatants were stored at −80° C. Protein concentration was assayed with Bradford reagent using BSA as a standard and quantitated with a spectrophotometer. Telomerase activity was detected by a PCR-based assay TRAPEZE Detection Kit (Chemicon). Two microliters of cell extract at 750 μg/μL was added to a total volume of 50 μL PCR reaction mix containing TRAP Reaction Buffer, dNTPs, substrate oligonucleotide, telomerase primer, internal standard primer, and Taq polymerase. As positive control, 2 μL of murine embryonic stem cell (mESC) extract was added to the reaction mix, and CHAPS lysis buffer alone and heat inactivated telomerase were used as negative control for each experimental sample. Each sample was incubated at 30° C. for 30 minutes for telomerase extension, followed by PCR amplification. For karyotyping, proliferating cells were incubated in culture with 0.1 μg/mL KaryoMAX Colcemid (Invitrogen) for 3-4 hours before they were re-suspended in hypotonic solution (0.075 M KCI) and incubated at room temperature for 10 minutes. Cells were then resuspended in cold fixative (3:1 methanol: acetic acid) and stored at 4° C. for at least 30 minutes. Following washing with fixative, cells were applied to clean glass slides and air dried. Metaphase chromosomes were prepared and karyotypes created using an Applied Spectral Imaging Band View digital imaging system. After 15 passages, PGS cells demonstrated high telomerase activity (FIG. 6) and normal karyotype (40, XY) (FIG. 7) as determined by cytogenetic analysis.

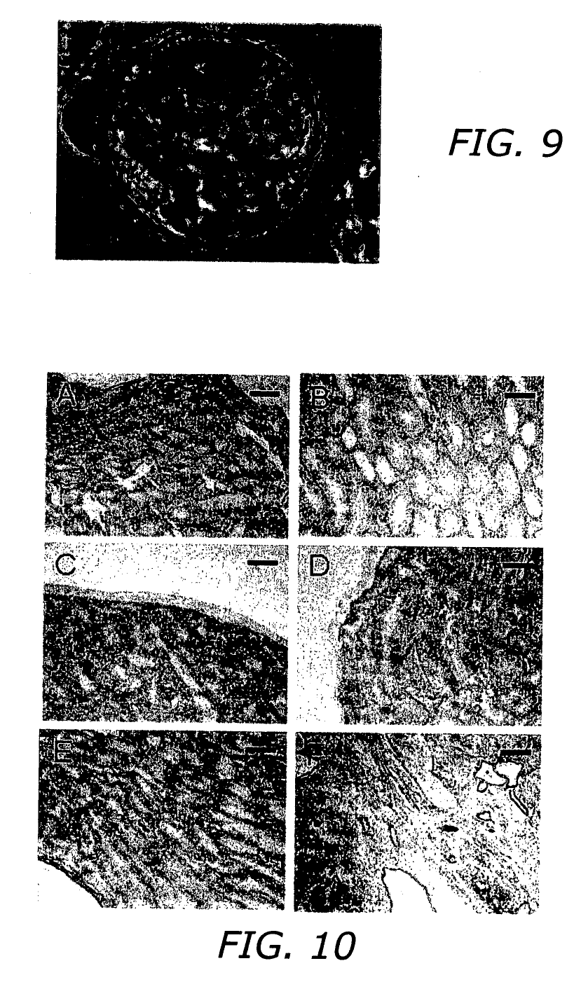

To define pluripotency, the ability of PGS cells to form chimeras in vivo was investigated. Undifferentiated Oct-4-GFP-positive cells isolated from all three PGS cell lines were microinjected (15-20 cells each) into embryos generated from albino CD-1 mice. Incorporation of GFP positive cells was observed in inner cell mass of the majority of the blastocysts injected with the PGS cells (FIG. 9). The embryos were subsequently implanted into the uterus of the pseudopregnant foster mice (a total of 234 implantations). Some of the recipient animals were sacrificed at 12.5 days post conception (dpc), the developing embryos retrieved and embryonic tissue, including the brain, heart and liver that represent the three germ layers were isolated for determination of the presence of GFP DNA as indications of chimera formation. The majority of recipient mice were allowed to carry pregnancy to term and GFP DNA was determined in different neonatal tissues using PCR amplification. GFP DNA was observed in brain (22%), heart (36.4%) and liver (25%) of the chimeric pups (FIG. 8). GFP DNA was also found in testes of 47% of the chimeric pups suggesting that PGS cells are capable of germ line transmission (Table 5).

TABLE 5

| | No. of | | Contribution of chimeric | | | |
|---|---|---|---|---|---|---|
| No. of embryos | pups | Total No. of | cells in different tissues | | | |
| transplanted | born | pups examined | Brain | Heart | Liver | Testis |
| 234 | 85 | 77 | 17/77 | 28/77 | 19/77 | 16/34 |
| | (36.3) | | (22) | (36.4) | (25) | (47) |

PGS cells were examined for their ability to form teratomas in vivo by subcutaneous, intramuscular or seminiferous tubular injections in nude mice (Harlan USA). As positive controls for teratoma formation, ES cells were injected in some mice. For subcutaneous or intramuscular injections, approximately 1×10⁶ cells were injected. For seminiferous tubular microinjections, approximately 2×10⁵ cells were injected through the efferent duct. Mice were sacrificed six weeks later, and tissues were harvested for morphological and histological analysis. The results showed that PGS cells did not form teratomas in the testis six weeks after transplantation (0/20). In contrast, formation of teratomas was observed in all recipient mice (6/6) which received undifferentiated ES cell transplantation (FIG. 10E-F).

Since the PGS cells are originated from the testis, their ability to differentiate into germ line cells was further examined by spermatogonial transplantation. This method allows spermatogonial stem cells to recolonize the empty seminiferous tubules of infertile animals and differentiate into mature sperm. Freshly isolated cells were sorted for c-kit and cells from the established PGS lines were transplanted into busulfan-treated nude mice testes. Six weeks after transplantation, only freshly isolated c-kit negative cells initiated spermatogenesis in the recipient mice (FIG. 10B, D); no spermatogenesis was observed after transplantation of the c-kit positive cells or unsorted PGS cells (FIG. 10C, E).

### Example 5

### Therapeutic Reprogramming of Murine Germ-Line Stem Cells into Cardiac Cells with PM-3 Medium

Murine primordial sex cells were isolated as described in Examples 1 and 2. The cells were then plated out at a density of 200,000 cells/3.8 cm² in PM-3 medium (Table 6) and incubated overnight. This day is designated Day 0.

TABLE 6
PM-3 Medium

DMEM (Specialty Media SLM-220B)
12% FBS (Hyclone)
2 mM L-glutamine (Invitrogen 35050-061)
0.1 mM MEM non-essential amino acids (Invitrogen 11140-050)
1X penicillin/streptomycin (Invitrogen 15070-063)
0.1 mM β-mercaptoethanol (Sigma M7522)
20 ng/mL rhEGF
10 ng/mL rhFGF2
2 ng/mL rhGDNF
1000 units/mL ESGRO ® (mouse LIF)

PM-3 medium is specifically designed for the differentiation and expansion of PSCs and therapeutically reprogrammed PSCs into specific lineage cells. The PM-3 media described above is an exemplary embodiment of the therapeutic reprogramming method of the present invention. It will be understood by persons skilled in the art that the concentrations of the components of PM-3 can vary and still achieve the intended results. In an embodiment of the present invention, the concentration of rhEGF can range from approximately 10 ng/mL to approximately 40 ng/mL, the concentration of rhFGF2 can range from approximately 10 ng/mL to approximately 40 ng/mL, the concentration of rhGDNF can range from approximately 2 ng/mL to approximately 40 ng/mL and the concentration of ESGRO® mouse LIF can range from approximately 1,000 units/mL to approximately 10,000 units/mL.

The following day (Day 1), the floating cells are collected and plated onto 0.1% gelatinized plates and cultured in appropriate medium (PM-3). The cells are 8-10 microns in size, round with appearance of pseudopods and non-adherent.

Cells cultured in PM-3 begin to change morphology at approximately day 8. This is the phase 1 of therapeutic reprogramming wherein the cell divides in distinct colonies and is approximately 10-15 micron in size, and is adherent. In addition cells began to appear longer and tapered at the ends, 15-20 microns in size. By day 8, therapeutic reprogramming steps 1-4 have already occurred, in medium PM-3 the therapeutic reprogramming of a germ line stem cell into a therapeutic cell that is capable of differentiation occurs much faster than in PM-1.

### Example 6

### Differentiation of Therapeutically Reprogrammed Pluripotent Germ Stem Cells

To determine whether PGS cells can be differentiated into other phenotypes, methods which induce differentiation of ES cells in vitro were used. To generate embryoid bodies (EBs), PGS colonies were dissociated with collagenase and plated in non-adhesive culture plates in PM-1 medium containing 15% FBS (Hyclone) for 4 days. In some experiments EBs were formed in hanging drops. They were then cultured in the serum-free N1 medium for neural selection: DMEM/F12 (Invitrogen, Carlsbad, Calif.) supplemented with ITS (insulin, 10 mg/L; transferring, 6.7 ng/L; selenium, 5.5 mg/L) and fibronectin (50 μg/mL). After 5-7 days, EBs were transferred to gelatin-coated culture plates in N2 medium (DMEM/F12 with ITS, and supplemented with bFGF at 10 ng/mL) for expansion of neural progenitor cells. For differentiation into cardiomyocytes, EBs were formed and cultured for two weeks in the presence of different cardiogenic compounds including DMSO (0.06 M), (5′-aza-2′-deoxy-cytidine AZA, 5 mM) and (Cardiogenol-C) 25-50 μM, (Calbiochem, San Diego Calif.). During the differentiation process, the morphology of cells was analyzed and samples were taken both for gene expression analysis by RT-PCR and immunohistochemical staining.

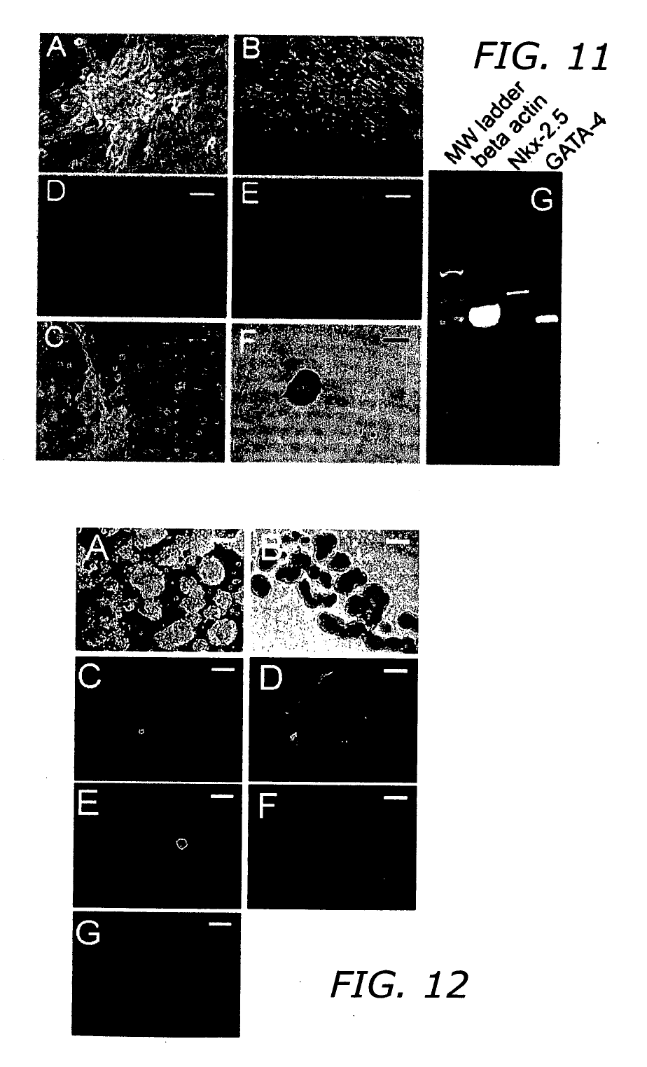

While undifferentiated PGS cells can be maintained and propagated in culture for more than 20 passages, spontaneous differentiation does occur. FIG. 11 depicts two populations in a PGS cell culture differentiated into cardiomyocytes as determined by the expression of troponin-1, a cardiac lineage marker expressed during early cardiogenesis (FIG. 11A-B and D-E). In the same culture, several cell populations exhibited rhythmic contractions that lasted up to several days (FIG. 11C). The expression of the cardiac-specific genes Nkx2.5 and GATA4 are also presented (FIG. 11G). Furthermore, spontaneous differentiation of PGS cells into adipocytes was observed in many cultures but in small numbers (FIG. 11F).

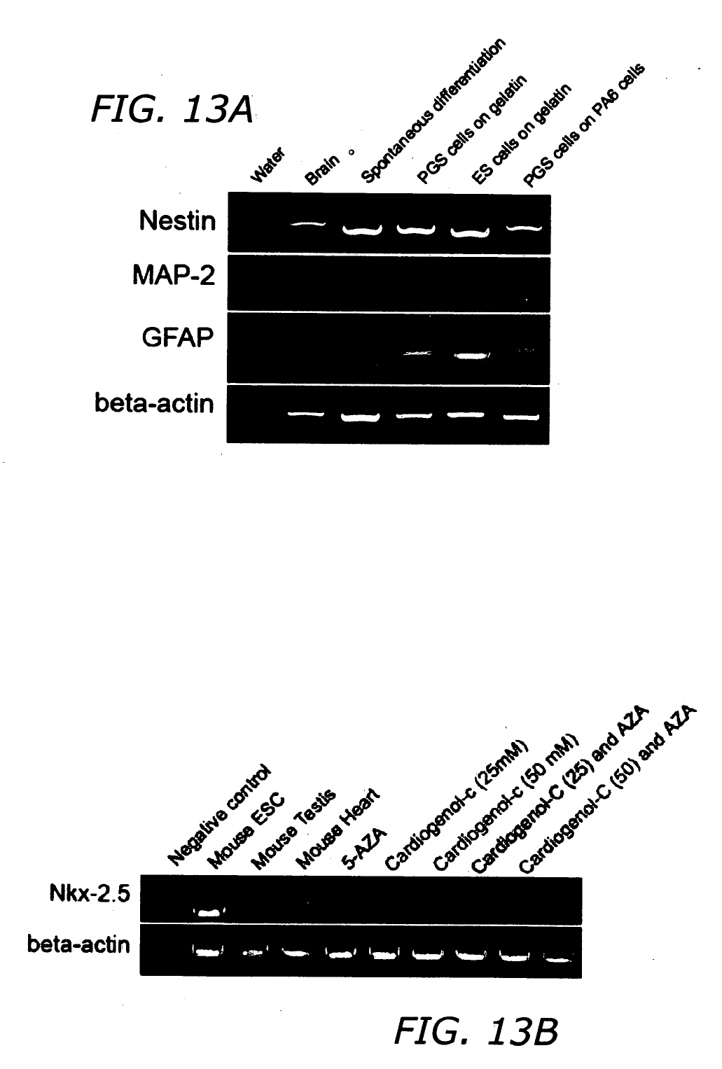

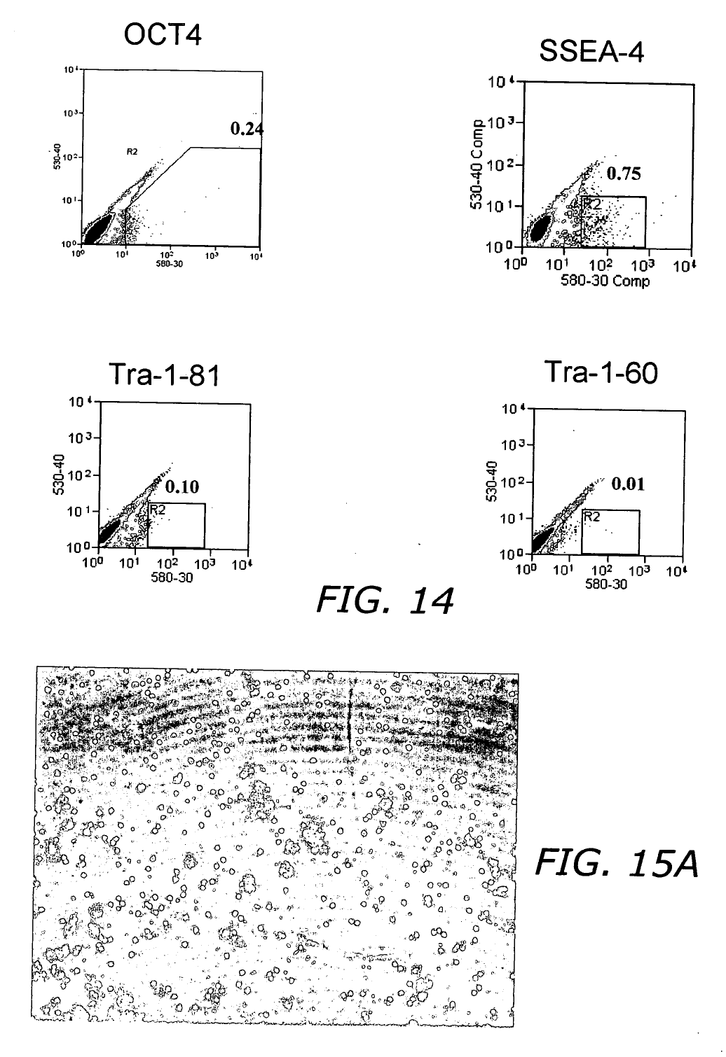

The ability of PGS cells to differentiate into multiple lineages in vitro was evaluated first by the formation of embryoid bodies (EBs) after cell aggregation. PGS cells were able to aggregate and form EBs both in suspension and in hanging drops. GFP signals were observed only for the first day after aggregation, indicating down regulation of Oct-4 and cell differentiation in the EBs (FIG. 12A, C). After EBs were subjected to neural selection by the N1 neural medium followed by expansion of neural progenitor cells in the N2 medium containing bFGF, cells dissociated from the EB expressed the neural progenitor marker, nestin (FIG. 12C). Some cells also stained positive for the astrocyte marker, GFAP (FIG. 12D), as well the neuronal marker neurofilament (FIG. 12E). The expression of these neural gene markers were confirmed by RT-PCR as shown in FIG. 13A. The PGS cell-derived EBs can also be induced to differentiate into cardiomyocytes after treatment with 5-aza-2-deoxy-cytidine (AZA) or cardiogenol-C. These phenotypes exhibited troponin-1 (FIG. 12C) and cardiac myosin heavy chain (FIG. 12G) as well as cardiac-specific gene marker, Nkx2.5 as shown in FIG. 13B.

### Example 7

### Isolation of Primordial Sex Cells from Human Testes

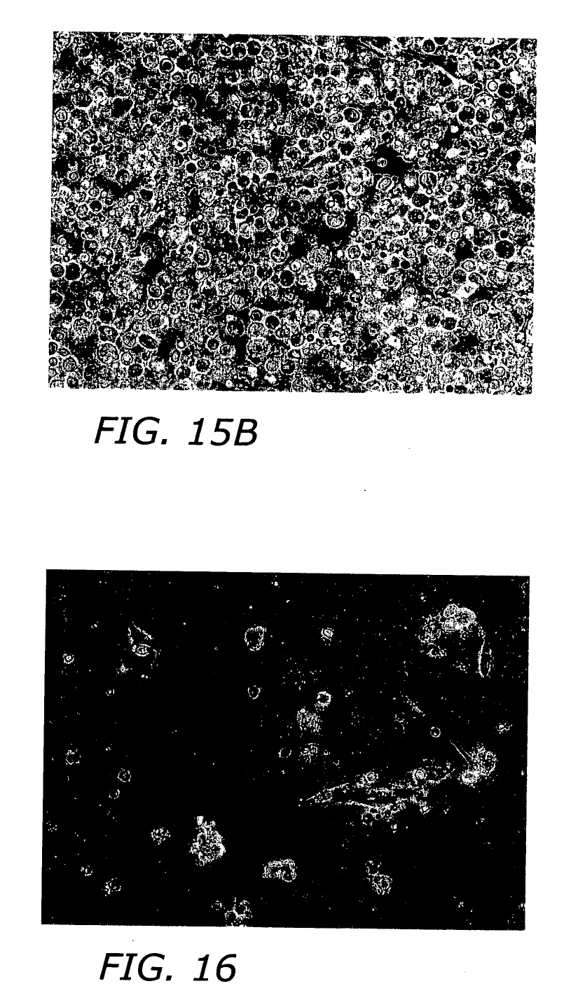

Testes were obtained under standard castration surgical procedures from male subjects who had been under ong-term hormone (estrogen) treatment. The testes were sanitized with 3% hydrogen peroxide for 5 min before they were excised and decapsulated. Testicular tissue was minced using fine scissors and subjected to a “one enzyme-one step” dissociation procedure. This procedure was modified from the mouse protocol as detailed in Example 1. Minced testicular tissues were transferred into culture medium (DMEM/F12) containing 1 mg/mL collagenase type 1 (Sigma) and 0.5 mg/mL DNase (Sigma). Digestion was performed at 37° C. for 30-60 min, depending on the tissue size, in a shaking water bath operated at 150 cycles/min. After neutralizing the enzyme activity with FBS (final concentration of 10%), dissociated cells were isolated from undigested tissues by passing the preparation through a 100-micron cell strainer (B-D BioScience). Cell suspensions were centrifuged at 400×g for 10 min at room temperature, and the pellet was reconstituted in PM-10 medium containing 1% FBS for differential adhesion for 24-72 hours, preferably 48 hours (FIG. 15A).

Additionally, testicular tissues can be subjected to a “two enzyme-two step” dissociation procedure. Testicular tissues were transferred into culture medium (DMEM/F12) containing 1 mg/mL collagenase type I and 0.5 mg/mL DNase. Digestion was performed at 37° C. for 10 min in a shaking water bath operated at 110 cycles/min. Interstitial cells were separated by sedimentation at unit gravity for 10 min and washed in DMEM/F12. A final digestion of the basal lamina components of the testicular tissue was carried out in a mixture of collagenase type I (1 mg/mL), DNase (0.5 mg/mL), and hyaluronidase (Sigma; 0.5 mg/mL) under the same conditions as for the first digestion step. The single-cell suspension obtained was washed successively with medium and PBS containing 1 mM EDTA (Sigma) and 0.5% fetal calf serum. The undigested remains of the tunica albuginea were eliminated by filtering the cell suspension through a 50 μm nylon mesh. All cells were kept at 5° C. throughout the procedure.

Additionally, testicular tissues can be subjected to a “three enzyme-two step” dissociation procedure. Minced testicular tissues were transferred into culture medium (DMEM/F12) containing 1.5 mg/mL collagenase type 1 and 0.5 mg/mL DNase. Digestion was performed at 37° C. for 15 min in a shaking water bath operated at 150 cycles/min. After neutralizing the enzyme activity with FBS (final concentration of 10%), dissociated cells were isolated from undigested tissues by passing the preparation through a 100-micron cell strainer. Cell suspensions were centrifuged at 400×g for 10 min at room temperature and the pellet was reconstituted in PM-10 medium containing 1% FBS for differential adhesion for 24-72 hours, preferably 48 hours (FIG.15B). The undigested tissues, primarily semiferous tubules with spermatogonial stem cells were dissociated again by a combination of three enzymes: 1.5 mg/mL collagenase type 1, 1.5 mg/mL hyaluronidase and 0.5 mg/mL trypsin (Sigma). Digestion was performed at 37° C. for 30-45 min in a shaking water bath operated at 150 cycles/min. After neutralizing the enzyme activity with FBS (final concentration of 10%), dissociated cells were isolated from undigested tissues by passing the preparation first through a 100-micron cell strainer and then a 40-micron cell strainer. Cell suspensions were centrifuged at 400×g for 10 min at room temperature and the pellet was reconstituted in 1-10 ml PM-100 medium at a concentration of 5-50 million cells/mL, preferably 10 million cells/mL. The cell suspension is loaded onto a chromatography glass column (45×450 mm, ChemGlass, Vineland, N.J.) with a funnel top. Cell fractionation was achieved by gravity sedimentation of cells over a 2-4% bovine serum albumin (BSA)-PBS solution at 4° C. for 2.5 hours. The fractionated cells were eluted at a flow rate of approximately 1-3 mumin, and 14 mL fractions were collected until the column was empty. Cell morphology in each fraction was examined microscopically.first under phase contrast and then Hoffman objectives to identify the fractions containing enriched spermatogonial stem cells for culture.

Alternatively, primordial sex cells from the testes can be isolated by dissecting the seminiferous tubules from the gonad, placing them into a dish containing 10 mL of 1 mM EDTA/DPBS(−) and incubated at room temperature for 15 minutes. The tunica albuginea is carefully dissected away from the seminiferous tubules. The seminiferous tubules are then placed into DPBS(−)/collagenase (1 mg/mL)/100 units of DNAase I and incubated at 37 C water bath with gentle shaking for 20-30 minutes. The digestion reaction is then quenched with equal volume of 20% FBS/DPBS(−). The cells were then washed two times with 20% FBS/DPBS(−).

Testicular cells isolated by any of the above-described methods can be further fractionated by fluorescence activated cell sorting (FACS). The dissociated testicular cells were suspended (5×10⁶ cells/mL) in PBS containing 0.5% FBS (PBS/FBS). The cells were then incubated with primary antibodies for 20 min on ice, washed twice with excess PBS/FBS, and used for FACS analysis. Primary antibodies include R-phycoerythrin (PE)-conjugated anti-α6-integrin, allophycocyanin (APC)-conjugated anti-c-kit, and biotinylated anti-αv-integrin and goat IgG anti-GDNF receptor. For experiments using secondary reagents, cells were further incubated for 20 min with APC-conjugated streptavidin to detect biotinylated antibody. All antibodies or secondary reagents were used at 5 μg/mL. Control cells are not treated with antibodies. After the final wash, the cells were resuspended (10⁷ cells/mL) in 2 mL PBS/FBS containing 1 μg/mL propidium iodide (Sigma), filtered into a tube through a 35 μm pore-size nylon screen, and kept in the dark on ice until analysis. The cells were sorted based on antibody staining and their relative granularity or internal complexity (side scatter, SSC). Cell sorting was performed by a dual-laser FACStar Plus (Becton Dickinson) equipped with 488-nm argon (200 mW) and 633-nm helium neon (35 mW) laser. An argon laser was used to excite PE and propidium iodide, and emissions were collected with a 575 DF 26 filter for PE and a 610 DF 20 filter for propidium iodide. A neon laser was used to excite APC, and emission was detected with a 675 DF 20 filter. Dead cells were excluded by eliminating propidium iodide-positive events at the time of data collection. Cells were sorted into 5 mL polystyrene tubes containing 2 mL of ice-cold DMEM supplemented with 10% FBS (DMEM/FBS). The α6-integrin^{hi}/SSC^{lo}/c-kit(−) population is further used for therapeutic reprogramming.

### Example 8

### Therapeutic Reprogramming of Human Germ-Line Stem Cells into Therapeutic Cells

Primordial sex cells were isolated as described in Example 7. Isolated human germ-line stem cells are then plated out in tissue culture dishes and incubated overnight at 37° C. to further remove adherent cells if necessary in a medium as disclosed in Tables 7 or 8.

TABLE 7

| | PM-10 Medium | PM-20 Medium | PM-100 Medium |
|---|---|---|---|
| Stem Cell Basal Medium | StemPro ®-34 | StemPro ®-34 | StemPro ®-34 |
| | Complete | Complete | Complete |
| L-glutamine | 2 mM | 2 mM | 2 mM |
| MEM vitamins | 1X | 1X | 1X |
| MEM non-essential amino | 1X | 1X | 1X |
| acids | | | |
| L-ascorbic acid | 0.1 mM | 0.1 mM | 0.1 mM |
| d-biotin | 10 μg/mL | 10 μg/mL | 10 μg/mL |
| β-estradiol | 50 ng/mL | 50 ng/mL | 50 ng/mL |
| progesterone | 60 ng/mL | 60 ng/mL | 60 ng/mL |
| bovine serum albumin | 5 mg/mL | 5 mg/mL | 5 mg/mL |
| Fraction VI | | | |
| DL-lactic acid | 1 μg/mL | 1 μg/mL | 1 μg/mL |
| pyruvic acid | 30 μg/mL | 30 μg/mL | 30 μg/mL |
| D-(+)-glucose | 6 mg/mL | 6 mg/mL | 6 mg/mL |
| sodium selenite | 30 nM | 30 nM | 30 nM |
| putrescine | 60 μM | 60 μM | 60 μM |
| transferrin (holo) | 50 μg/mL | 50 μg/mL | 50 μg/mL |
| bovine insulin | 20 μg/mL | 20 μg/mL | 20 μg/mL |
| penicillin/streptomycin | 1X | 1X | 1X |
| β-mercaptoethanol | 50 μM | 50 μM | 50 μM |
| recombinant human epidermal | 20 ng/mL | 20 ng/mL | 20 ng/mL |
| growth factor (rhEGF) | | | |
| recombinant human fibroblast | 1 ng/mL | 1 ng/mL | 110 ng/mL |
| growth factor 2 (rhFGF2) | | | |
| recombinant human glial cell | 10 ng/mL | 20 ng/mL | 10 ng/mL |
| derived neurotrophic factor | | | |
| (rhGDNF) | | | |
| 1,000 units/mL human l | 1000 U/mL | 1000 U/mL | 1000 U/mL |
| eukemia inhibitory factor (LIF) | | | |

TABLE 8

| | PM-5 Medium | PM-101 Medium |
|---|---|---|
| FBS (Hyclone) | 1% | 1% |
| Stem Cell Basal Medium | Stem Pro ®-34 | Stem Pro ®-34 |
| | Complete | Complete |
| L-glutamine | 2 mM | 2 mM |
| Minimal Essential Medium | 1X | 1X |
| (MEM) vitamins | | |
| MEM non-essential amino acids | 1X | 1X |
| L-ascorbic acid | 0.1 mM | 0.1 mM |
| d-biotin | 10 μg/mL | 10 μg/mL |
| β-estradiol | 50 ng/mL | 50 ng/mL |
| progesterone | 60 ng/mL | 60 ng/mL |
| bovine serum albumin Fraction VI | 5 mg/mL | 5 mg/mL |
| DL-lactic acid | 1 μg/mL | 1 μg/mL |
| pyruvic acid | 30 μg/mL | 30 μg/mL |
| D-(+)-glucose | 6 mg/mL | 6 mg/mL |
| sodium selenite | 30 nM | 30 nM |
| putrescine | 60 μM | 60 μM |
| transferrin (holo) | 50 μg/mL | 50 μg/mL |
| bovine insulin | 20 μg/mL | 20 μg/mL |
| penicillin/streptomycin | 1X | 1X |
| β-mercaptoethanol | 50 μM | 50 μM |
| recombinant human epidermal | 20 ng/mL | 20 ng/mL |
| growth factor (rhEGF) | | |
| recombinant human fibroblast | 2 ng/mL | 10 ng/mL |
| growth factor 2 (rhFGF2) | | |
| recombinant human glial cell | 20 ng/mL | 10 ng/mL |
| derived | | |
| neurotrophic factor (rhGDNF) | | |
| 1,000 units/mL human leukemia | 1000 U/mL | 1000 U/mL |
| inhibitory factor (LIF) | | |

StemPro®-34 Complete Medium (Invitrogren Corporation, Carlsbad, Calif.) is comprised of StemPro®-34 Serum Free Medium (SFM) and StemPro®-34 Nutrient Supplement and disclosed in U.S. Pat. No. 6,733,746 B2, the contents of which are herein incorporated by reference in their entirety, particularly Tables 1, 2 and 3 in columns 17-18. For the purposes of this disclosure, StemPro®-34 Complete Medium will be referred to as Stem Cell Basal Medium. Tables 2 and 3, which list the components of the StemPro-34 Complete Medium used in the cell culture medium of the present invention are reproduced below exactly as they are found in the '746 patent.

PM-10, PM-20 and PM-100 media were specifically designed for the culture and maintenance of human primordial sex cells (hPSC) and their initial reprogramming into pluripotent embryonic stem cell-like cells in a serum-free environment. The PM-10, PM-20 and PM-100 media described above are exemplary embodiments of the therapeutic reprogramming method of the present invention. It will be understood by persons skilled in the art that the concentrations of the components of PM-10, PM-20 and PM-100 media can vary and still achieve the intended results. In an embodiment of the present invention, the concentration of rhEGF can range from approximately 10 ng/mL to approximately 40 ng/mL, the concentration of rhFGF2 can range from approximately 1 ng/mL to approximately 120 ng/mL, the concentration of rhGDNF can range from approximately 2 ng/mL to approximately 40 ng/mL and the concentration of human LIF can range from approximately 1,000 units/mL to approximately 10,000 units/mL.

After differential adhesion for 24-72 hours, cell sorting by stem cell specific antibodies or sedimentation fractionation, the isolated testicular cells were replated on fresh tissue culture dishes coated with 0.1 %-0.2% gelatin and/or feeder cells (mouse or human fibroblasts) and cultured in PM-10, PM-20 or PM-100 media. At this stage the cells were approximately 8-10 micron in size and non-adherent (FIG. 15A and B). The cells were cultured for a total of 3-17 days before continuing.

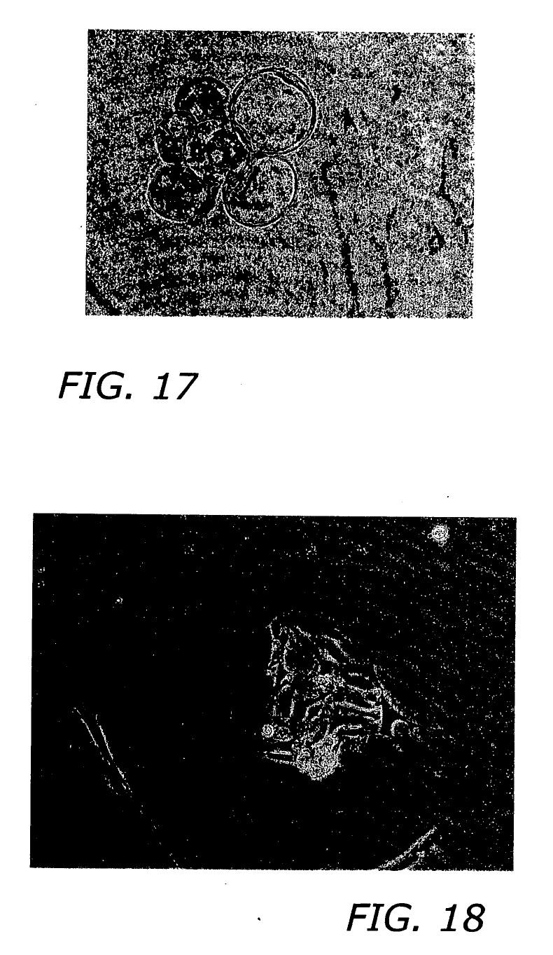

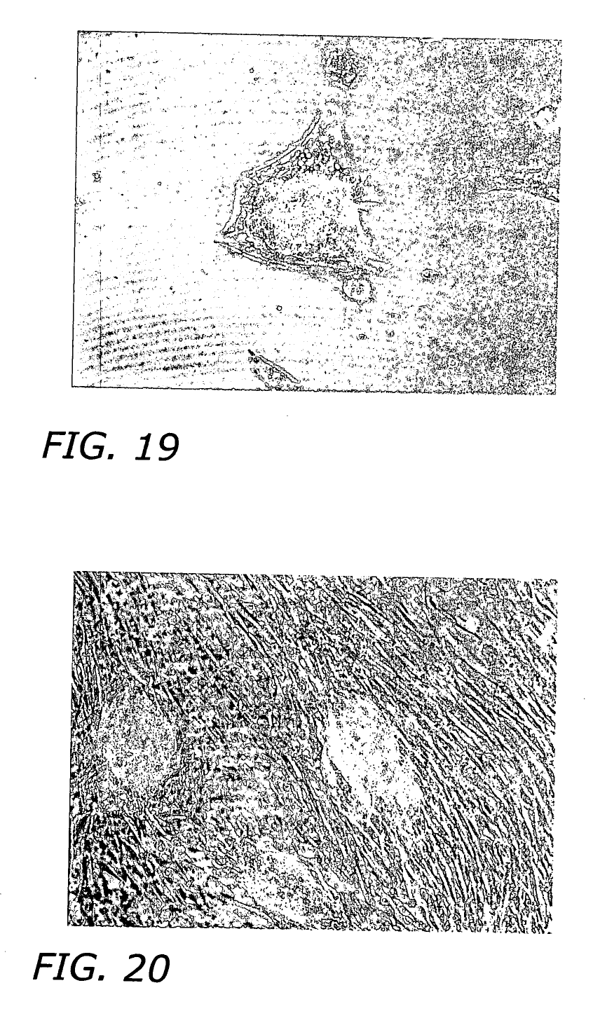

The non-adherent germ line stem cells next attach (FIG. 16) and convert into “egg-like” structures (FIG. 17). The human germ-line stem cells were cultured for an additional 3-17 days when “grape-like” clusters of cells appear in the culture media (FIG. 18). The appearance of these grape-like clusters can also be induced by culturing cells in human embryo culture medium (Complete Blastocyst Medium with SSS, Irvine Scientific catalog #9930). The cells were continued to be cultured in PM-10, PM-20 or human embryo culture medium until the cells begin to form tightly compact colonies (FIG. 19).

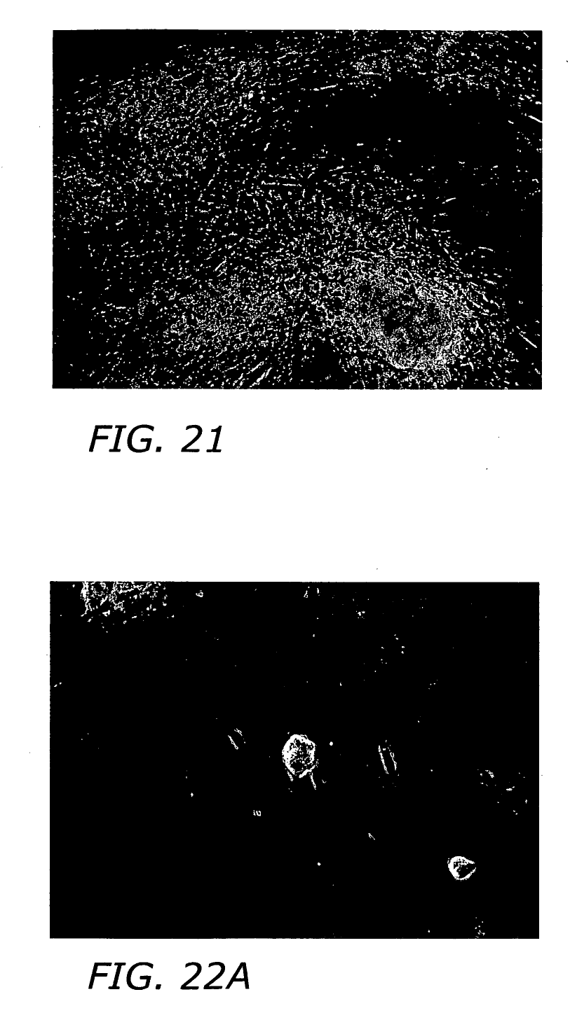

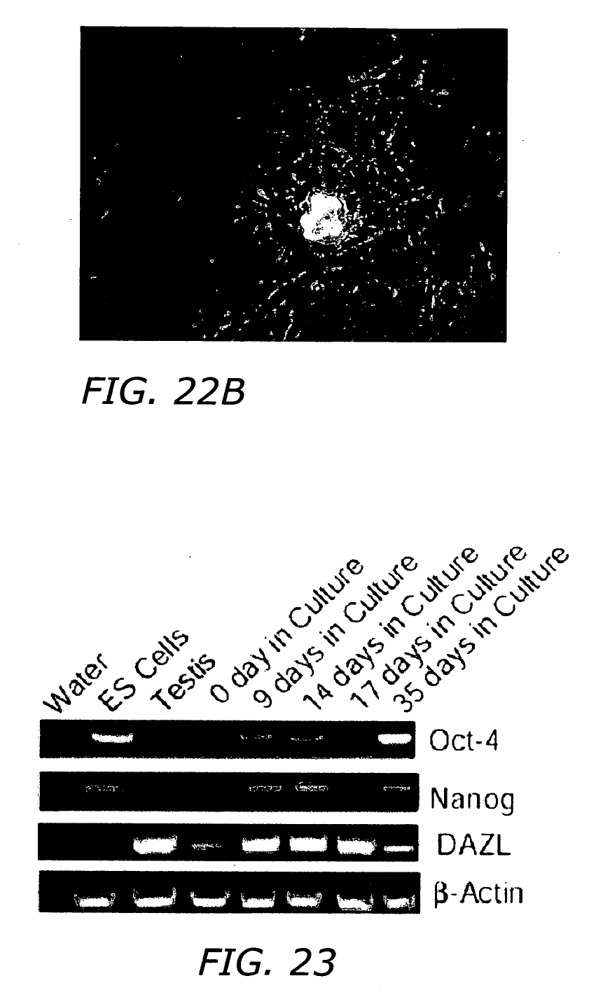

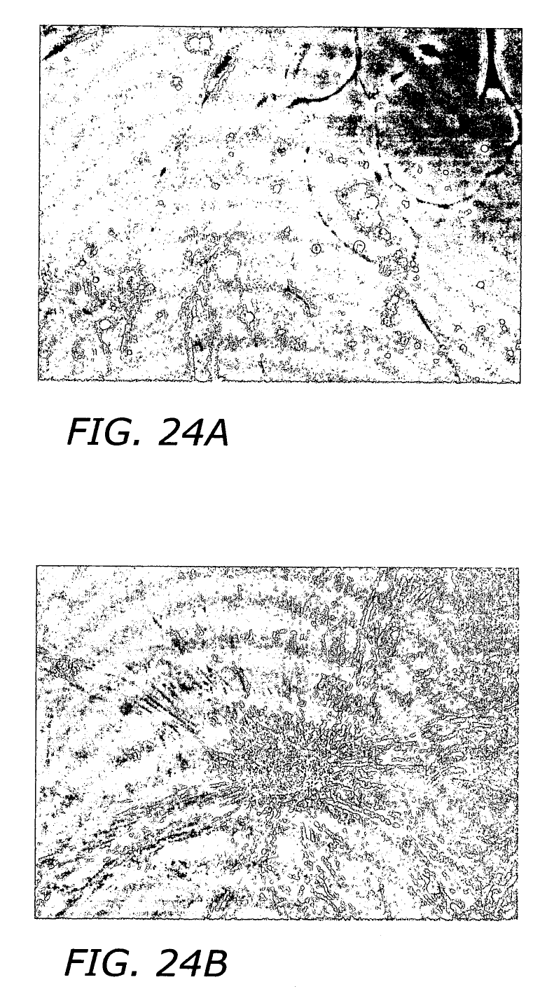

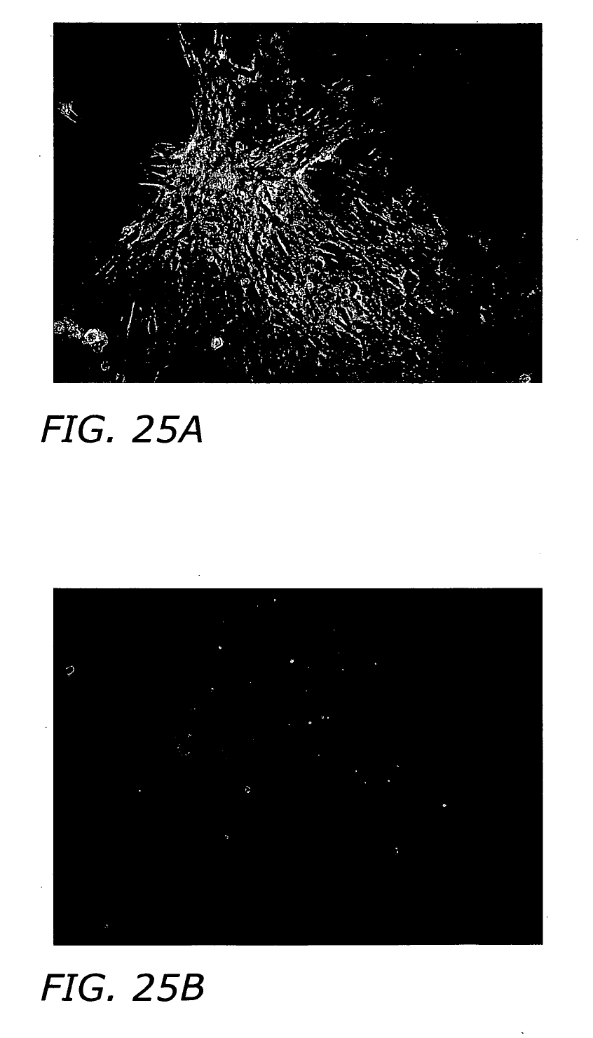

The colonies were then collected and dissociated into a 6 cm tissue culture dish optionally containing mitomycin C inactivated primary embryonic fibroblasts (human or mouse) in the final step in the conversion pathway of a human germ-line stem cell into a pluripotent germ line stem (PGS) cell that is suitable for use for cell-based regenerative therapies. The resultant PGS cells were characterized by forming very distinct compact colonies (FIG. 20) that resemble colonies formed by embryonic stem cells. These cells were also characterized by the expression of human-specific markers (staining with anti-human mitochondrial antibody [Chemicon MAB 1273], FIG. 21), Oct-4 (Chemicon MAB 4305, FIG. 22A) and Nanog (Bethyl A300-398A, FIG. 22B). Reprogramming is a controlled process involving the expression of Oct-4, a signature gene expressed in pluripotent stem cells. Oct-4 is down-regulated as pluripotent cells differentiate and up-regulated as differentiated cells regain their pluripotent potential. As mouse testicular stem cells are reprogrammed to pluripotent PGS colonies, the expression of Oct-4 re-appeared (Example 4). Thus tracking the dynamics of Oct-4 expression is used as an indication of reprogramming in human testicular stem cell culture. It has been well documented that adult human testes do not expressed Oct-4 (FIG. 23, lane 3). Cells isolated from testicular tissues and processed through enzyme dissociation, differential adhesion or fractionation also do not expression Oct-4 (FIG. 23, lane 4). In contrast, Oct-4 expression was up-regulated between 9 and 35 days of culture (FIG. 23, lanes 5-8). This evidence demonstrates that adult human testicular stem cells have been therapeutically reprogrammed, that is they regained pluripotentiality as evidenced by expression of Oct-4 during culture in a serum-free medium (PM-10, PM-20 or PM-100 medium). Several pluripotent markers, including Nanog, (FIG. 23), Dppa5, Sox2, alkaline phosphatase, SSEA-3, SSEA-4, TRA-1-60 and TRA-1-81, were also found to be expressed after reprogramming. The identity of human germ-line cells was confirmed by staining the cells with antibodies to the germ-line specific marker DAZL (FIG. 23).

Reprogrammed, pluripotent human stem cells were expanded by three protocols: a two-dimensional serum-free protocol, a two-dimensional serum-containing protocol and a three-dimensional serum-free protocol. In the two-dimensional serum-free protocol, isolated and reprogrammed testicular stem cells were dissociated by collagenase type 1 (1 mg/mL) or trypsin (1 mg/mL) and re-plated onto a gelatin (0.1-0.2%)-coated surface or feeder cells (inactivated mouse or human embryonic fibroblasts). Cells were cultured and expanded in PM-10, PM-20 or PM-100 medium (FIG. 20). Expanded cell colonies were propagated, frozen, or differentiated into specific phenotypes.

In the two-dimensional serum-containing protocol, cells were cultured and expanded in PM-10 containing 15-20% FBS, or in PM-5 or PM-101 medium. Expanded cell colonies were manually picked or-dissociated by collagenase type 1 (1 mg/mL) or trypsin (1 mg/mL), plated onto gelatin-coated dishes (0.1-0.2%), onto plastic culture flasks, MEF feeders, Matrigel® matrix, or fibronectin-coated dishes or coverslips. Expanded cell colonies were propagated, frozen, or differentiated into specific phenotypes.

In the three-dimensional serum-free protocol, cells after isolation, differential adhesion, and/or BSA fractionation were cultured in PM-100 medium in suspension in a bioreactor with spinning culture. The cells were placed in a 500 mL or 250 mL CytoSpin flask (VWR) and cultured in 400 mL or 200 mL of PM-100 medium, respectively. The spinning was accomplished by placing the flask on a stirrer with a spinning speed set at 50-70 cpm at 34-37° C. in an incubator. Samples were collected at frequent intervals to evaluate cell viability, cell number, pluripotent markers such as Oct-4 and Nanog expression and cell expansion. Medium in the bioreactors were changed every 5-7 days by centrifugation and reconstitution in fresh medium. Expanded cell colonies were propagated, frozen, or differentiated into specific phenotypes.

### Example 9

### Differentiation of Therapeutically Reprogrammed Human Pluripotent Germ Line Stem Cells into Cardiac Cells

Therapeutically reprogrammed human post-natal stem cells from Example 8 can be differentiated into a number of different cell types. Primordial sex cells were isolated as described in Example 7. The cells were then plated out onto 6 cm tissue culture dishes previously treated with 0.1% gelatin and cultured for 30 days in a standard CO₂ cell culture incubator at a temperature range of 34° C. to 42° C. in media containing aza-2′-deoxycytidine which was changed every other day for 30 days.

Aza-2′-deoxycytidine was added to PM-1, PM-3, PM-10 or PM-20 media at a concentration of approximately 0.1 μM to 9.0 μM. PM-1 medium is described in Example 3, PM-3 medium is described in Example 6 and PM-10 and PM-20 media are described in Example 8. The aza-2′-deoxycytidine-containing media described herein is an exemplary embodiment of the therapeutic reprogramming method of the present invention. It will be understood by persons skilled in the art that the concentrations of the components of the aza-2′-deoxycytidine-containing media can vary and still achieve the intended results. For the purposes of this example, the aza-2′-deoxycytidine-containing media is PM-1-AZA.

After approximately 11 days in PM-1-AZA culture media, the suspension germ-line stem cells undergo therapeutic reprogramming and attach to the plate and form colonies (FIG. 24A). After approximately 22 days in PM-1-AZA culture media, the cells had formed distinct colonies (FIG. 24B)

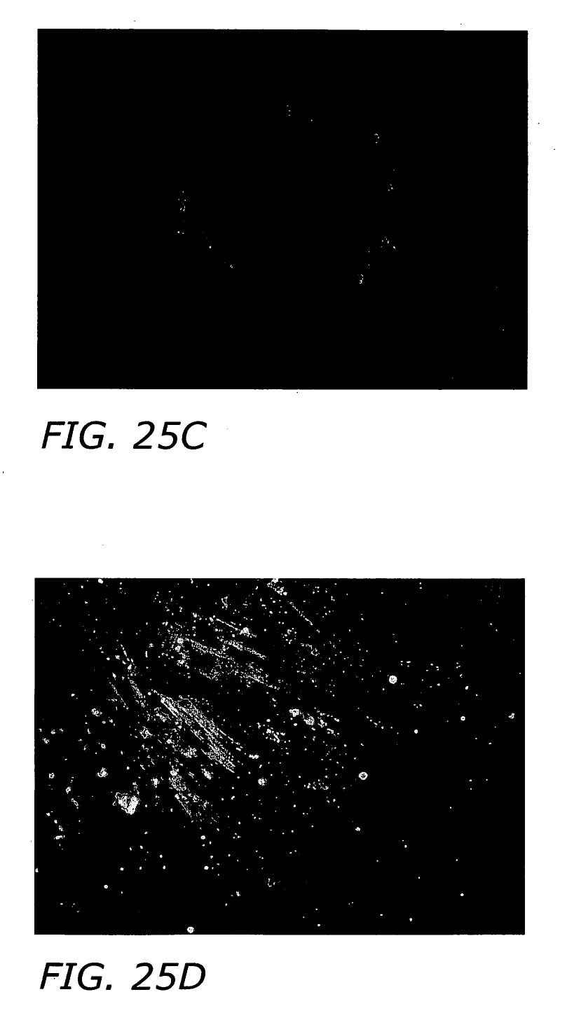

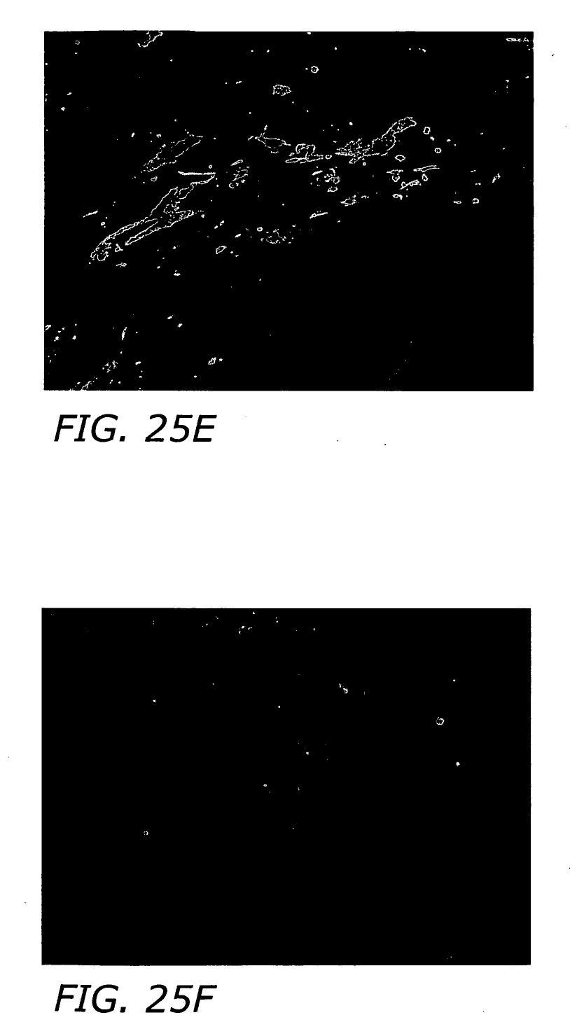

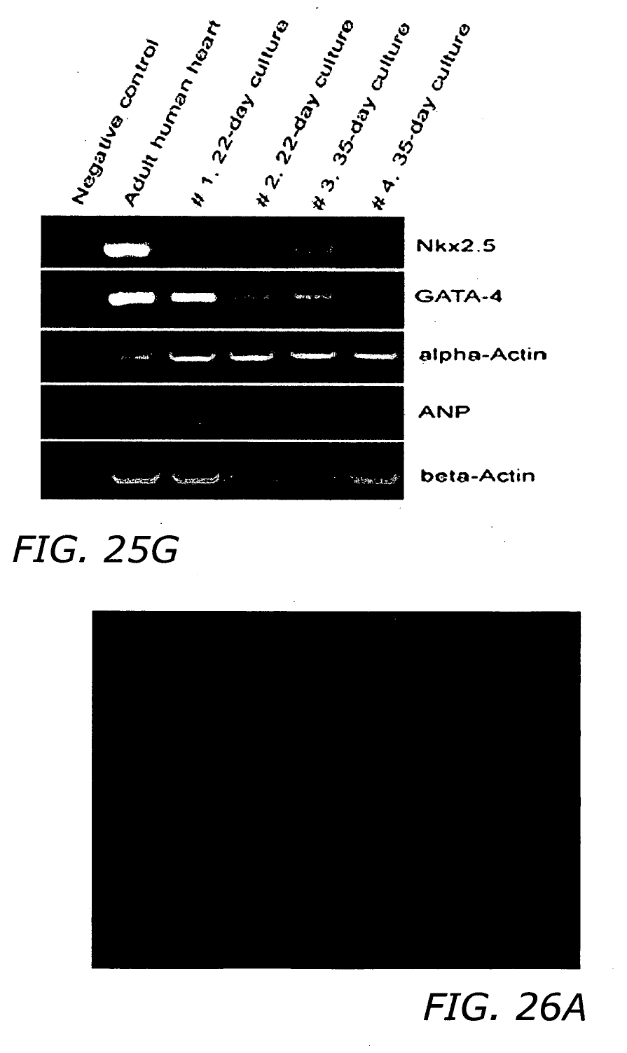

After approximately 35 days in culture with PM-1-AZA media, the suspension germ-line stem cells have differentiated and stain positive for the cardiac marker cardiac troponin 1 (FIG. 25A-C), cardiac alpha actin (FIG. 25D), desmin (FIG. 25E) and cardiac myosin (FIG. 25F) and expressed cardiomyocyte specific genes including Nkx2.5 and GATA-4 (FIG. 25G).

### Example 10

### Differentiation of Therapeutically Reprogrammed Human Pluripotent Germ Line Stem Cells into Tissue Specific Cells

Isolated therapeutically reprogrammed human germ line stem cells from Example 8 were cultured in PM-SHH media (Table 9) at 34° C. for 24 days before they were identified for expression of the glial cell marker, glial fibrillary acidic protein (GFAP) by fluorescence immunocytochemistry using a monoclonal anti-GFAP antibody (B-D BioScience Cat. # 556329). No foreign cells were introduced during the entire culture period. The media was refreshed every other day.

TABLE 9

| | PM-SHH Medium |
|---|---|
| Stem Cell Basal Medium | Stem Pro ®-34 Complete |
| L-glutamine | 2 mM |
| MEM vitamins | 1X |
| MEM non-essential amino acids | 1X |
| L-ascorbic acid | 0.1 mM |
| d-biotin | 10 μg/mL |
| β-estradiol | 50 ng/mL |
| progesterone | 60 ng/mL |
| bovine serum albumin Fraction VI | 5 mg/mL |
| DL-lactic acid | 1 μg/mL |
| pyruvic acid | 30 μg/mL |
| D-(+)-glucose | 6 mg/mL |
| sodium selenite | 30 nM |
| putrescine | 60 μM |
| transferrin (holo) | 50 μg/mL |
| bovine insulin | 20 μg/mL |
| penicillin/streptomycin | 1X |
| β-mercaptoethanol | 50 μM |
| rhEGF | 20 ng/mL |
| rhFGF-2 | 5 ng/mL |
| rhGDNF | 10 ng/mL |
| rhLIF | 1000 U/mL |
| recombinant human platelet-derived growth | 20 ng/mL |
| factor-BB (rhPDGF-BB) | |
| Transforming growth factor β3 (TGF-β3) | 4 ng/mL |
| recombinant human fibroblast growth factor | 75 ng/mL |
| 8 (rhFGF-8) | |
| recombinant human sonic hedgehog (SHH) | 150 ng/mL |

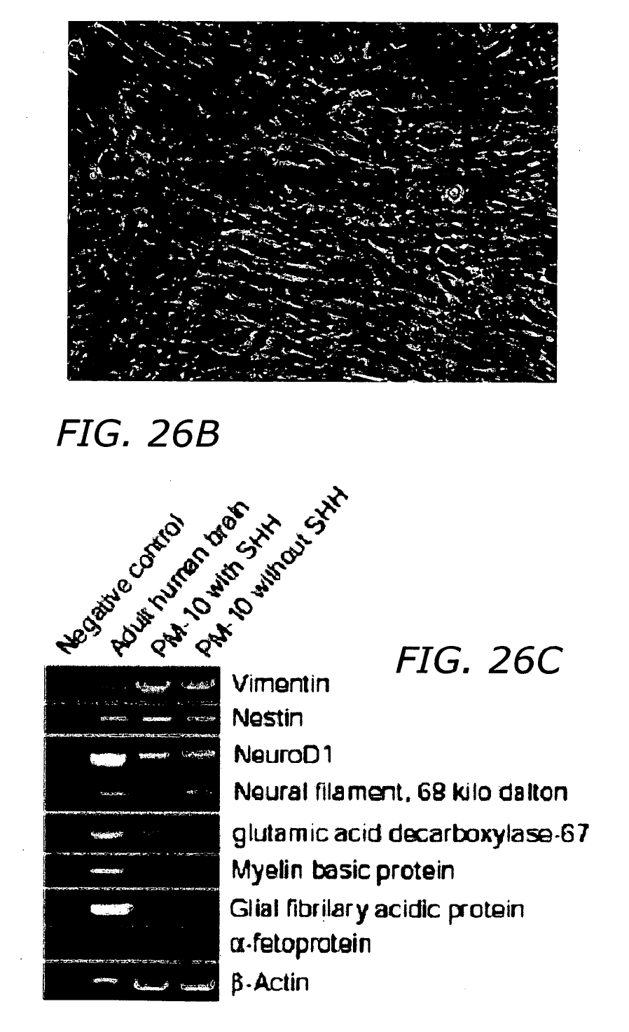

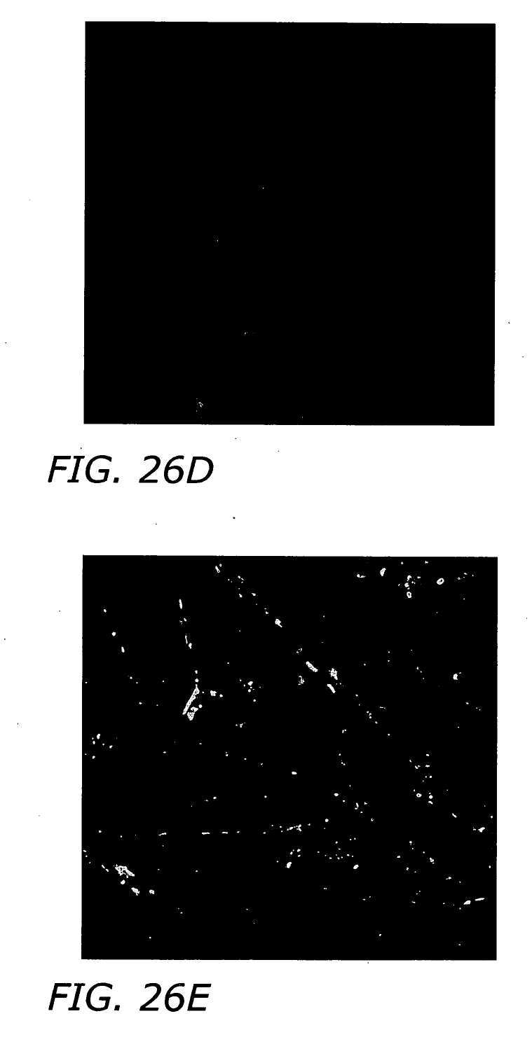

In the stained cell populations, GFAP⁺ cells were observed only in populations cultured in PM-SHH media (FIG. 26A) but not in PM-1 media (FIG. 26B). These results provide evidence that isolated human gonadal stem cells can be directed to differentiate into neural cells, including GFAP⁺ glial cells, in vitro. Since there is no known evidence for the existence of glial cells in the adult testis, the GFAP glial cells demonstrated above differentiated from gonadal stem cells (pluripotent cells) via therapeutically reprogramming.

In addition to astroglial differentiation, reprogrammed pluripotent testicular stem cells can also be differentiated into neuronal cells in the PM-SHH medium as evidenced by the expression of neuron-specific markers, including MAP-2, NF-68, and GAD-67 (FIG. 26C). During reprogramming and expansion, co-culture of testicular stem cells with embryonic mouse pre-adipocytes (mouse PA 6 cells, which are isolated from skull bone marrow; PA6 cells are purchased from Rikins, Japan) results in the differentiation of testicular stem cells into neurons as well as astroglial cells. Specific neuronal cell types were identified after differentiation, including GABAergic neurons (GAD-67 expression) and dopaminergic neurons (stained positive by antibodies to tyrosine hydroxylase).

Oligodendroglial cells were differentiated from reprogrammed pluripotent testicular stem cells as following. For myelin basic protein (MBP) expression (FIG. 26D), testicular stem cells isolated and propagated by the two-dimensional serum-containing method were plated onto fibronectin-coated coverslips in N2 medium with, 10 ng/mL bFGF, 10 ng/mL EGF and 1 ng/mL PDGF. Control cells were cultured on 0.2% gelatin-coated coverslips in PM-10 medium with 2% FBS without the growth factors. For determining galactocerebroside-C (Gal-C) and proteolipid protein (PLP) expression (FIG. 26E), testicular stem cells isolated and propagated by the two-dimensional serum-containing method were plated onto fibronectin-coated coverslips in DMEM low glucose with GlutaMax with 40% MCDB-201 media (Sigma) and supplemented with ITS+LA−BSA (10 μg/mL insulin from bovine pancreas, 5.5 μg/mL human transferrin (substantially iron-free), 5 ng/mL sodium selenite, 0.5 mg/mL bovine serum albumin and 4.7 μg/mL linoleic acid), 1 nM dexamethasone, 100 μM ascorbic acid and treated with 200 ng/mL SHH and 100 ng/mL FGF-8 for 3 days and then switched to N2 supplements with 20 ng/mL BDNF. Control cells were cultured without the addition of SHH and FGF-8 on 0.2% gelatin-coated coverslips in DMEM low glucose with GlutaMax with 2% FBS, MCDB-201 40% and supplemented with ITS+LA−BSA, 1 nM dexamethasone and 100 μM ascorbic acid.

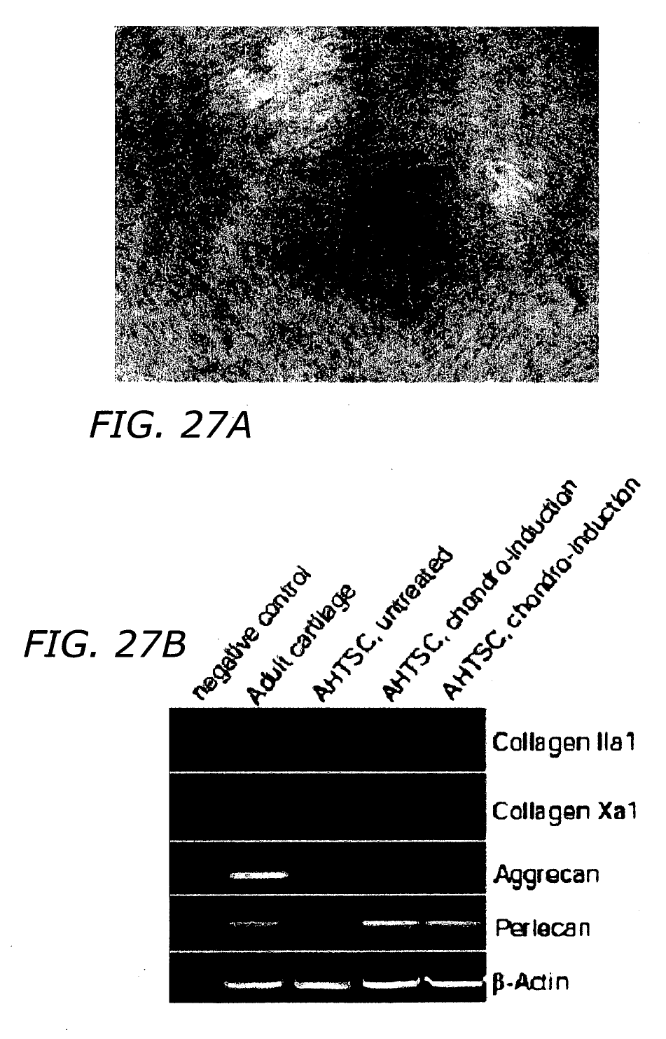

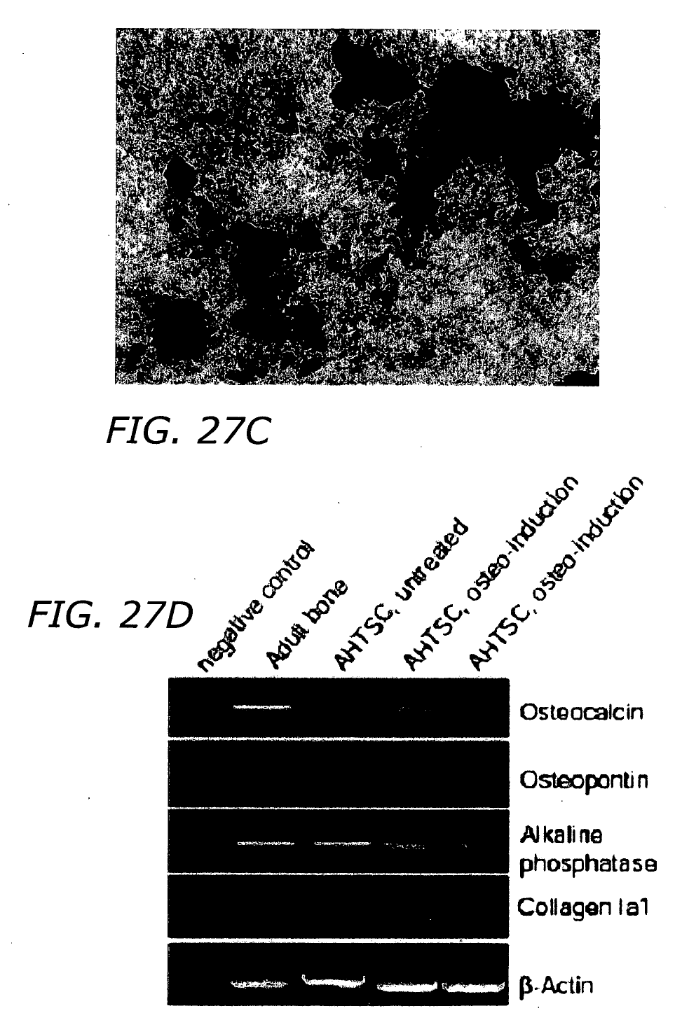

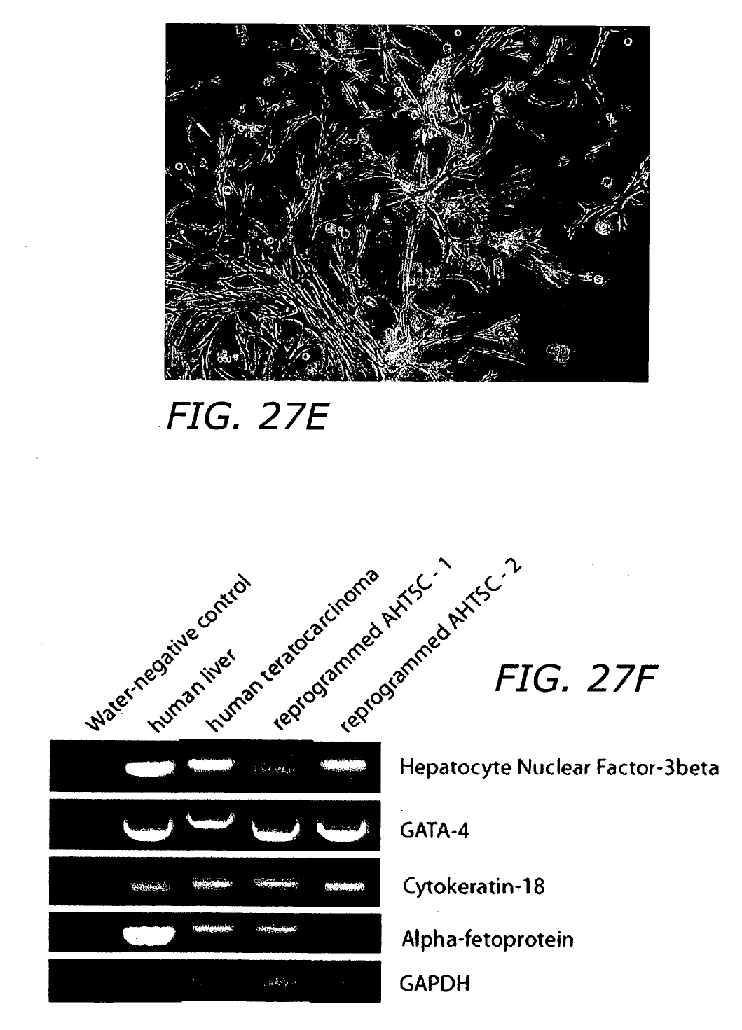

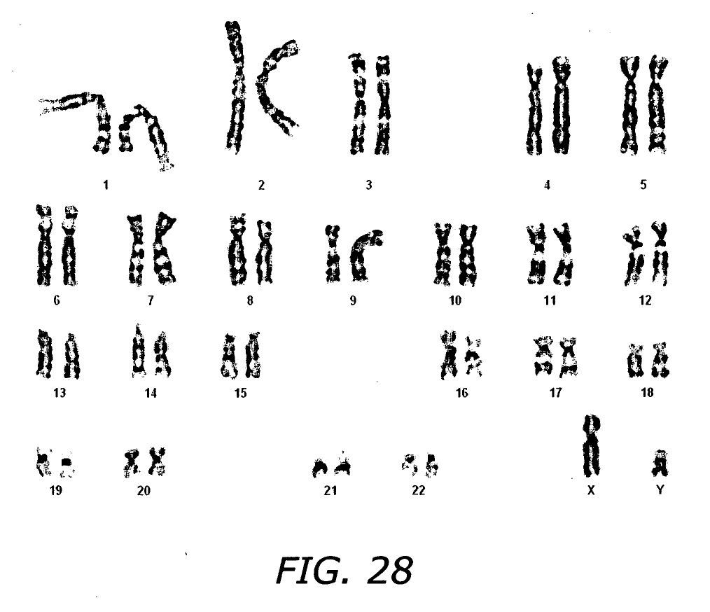

In addition to cardiac and neural differentiation, reprogrammed pluripotent testicular stem cells can also be differentiated into chondrocytes (FIG. 27A-B), osteocytes (FIG. 27C-D) and hepatocytes (FIG. 27E-F).

For chondrocyte differentiation, testicular stem cells isolated and propagated by the two-dimensional serum-containing method were plated onto 0.2% gelatin-coated plates in SingleQuots® medium (Cambrex PT4124) with 20% FBS and 10 ng/mL TGF-3β added to the medium just before the medium change. Control cells were cultured in DMEM low glucose with L-glutamine and penicillin/streptomycin supplemented with 20% FBS.

For osteocyte differentiation, testicular stem cells isolated and propagated by the two-dimensional serum-containing method were plated onto 0.2% gelatin-coated plates in DMEM low glucose with L-glutamine and penicillin/streptomycin supplemented with 20% FBS and the addition of 100 nM dexamethasone, 0.25 mM ascorbic acid and 10 mM B-glycerolphosphate. Medium change was every 2-3 days. Control cells were plated on 0.2% gelatin-coated plates in DMEM low glucose with L-glutamine and penicillin/streptomycin supplemented with 20% FBS.

For hepatocyte differentiation, testicular stem cells isolated and propagated by the two-dimensional serum-containing method were plated onto Matrigel®-coated plates in DMEM low glucose with GlutaMax with 5% FBS, 40% MCDB-201 and supplemented with ITS+LA−BSA, 1 nM dexamethasone, 100 μM ascorbic acid, 10 ng/mL FGF-4 and 20 vng/mL HGF.(hepatocyte growth factor). Control cells were kept in DMEM with 2% FBS on 0.2% gelatin-coated plates.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A therapeutic reprogramming method comprising:
isolating a human post-natal stem cell;
contacting said human post-natal stem cell with a medium comprising stimulatory factors which induce development of said human post-natal stem cell into a therapeutically reprogrammed cell wherein said human post-natal stem cell is therapeutically reprogrammed to a pluripotent human stem cell.

2. The therapeutic reprogramming method of claim 1 further comprising the step of differentiating said pluripotent human stem cells to yield a tissue specific cell.

3. The therapeutic reprogramming method of claim 1 wherein said human post-natal stem cell is a primordial sex cell.

4. The therapeutic reprogramming method of claim 3 wherein said primordial sex cell is a diploid germ cell isolated from a male or female post-natal source.

5. The therapeutic reprogramming method of claim 3 wherein said primordial sex cell is a spermatogonial stem cell.

6. The therapeutic reprogramming method of claim 1 wherein said medium comprises PM-10 medium.

7. The therapeutic reprogramming method of claim 1 wherein said tissue specific cell is selected from the group consisting of cardiac myocytes, glia, neurons, hepatocytes, osteocytes and chondrocytes.

8. A cell culture medium for therapeutically reprogramming post-natal stem cells wherein said cell culture medium comprises:
a cell culture growth medium base;
a plurality of vitamins and minerals; and
a plurality of cell growth and maturation factors,

9. The cell culture medium of claim 8 wherein said post-natal stem cell is a human stem cell.

10. The cell culture medium of claim 8 wherein said medium does not contain serum.

11. The cell culture medium of claim 8 wherein said plurality of cell growth and maturation factors comprise factors selected from the group consisting of recombinant human epidermal growth factor (rhEGF), recombinant human fibroblast growth factor 2 (rhFGF-2), recombinant human glial cell derived neurotrophic factor (rhGDNF) and human leukemia inhibitory factor (LIF), platelet-derived growth factor BB (PDGF-BB), transforming growth factor-β3 (TGF-β3), fibroblast growth factor 8 (FGF-8) and sonic hedgehog (SHH).

12. The cell culture medium of claim 11 wherein said recombinant human epidermal growth factor is present at a concentration of between 10 ng/mL and 40 ng/mL.

13. The cell culture medium of claim 12 wherein said recombinant human epidermal growth factor is present at a concentration of 20 ng/mL.

14. The cell culture medium of claim 11 wherein said recombinant human fibroblast growth factor 2 is present at a concentration of between 1 ng/mL and 120 ng/mL.

15. The cell culture medium of claim 14 wherein said recombinant human fibroblast growth factor 2 is present at a concentration of between 1 ng/mL and 2 ng/mL.

16. The cell culture medium of claim 14 wherein said recombinant human fibroblast growth factor 2 is present at a concentration of between 10 ng/mL and 20 ng/mL.

17. The cell culture medium of claim 14 wherein said recombinant human fibroblast growth factor 2 is present at a concentration of 110 ng/mL.

18. The cell culture medium of claim 11 wherein said recombinant human glial cell derived neurotrophic factor is present at a concentration of between 2 ng/mL and 40 ng/mL.

19. The cell culture medium of claim 18 wherein said recombinant human glial cell derived neurotrophic factor is present at a concentration of 2 ng/mL.

20. The cell culture medium of claim 18 wherein said recombinant human glial cell derived neurotrophic factor is present at a concentration of between 10 ng/mL and 20 ng/mL.

21. The cell culture medium of claim 11 wherein said human leukemia inhibitory factor is present at a concentration of between 1,000 units/mL and 10,000 units/mL.

22. The cell culture medium of claim 21 wherein said human leukemia inhibitory factor is present at a concentration of 1,000 units/mL.

23. A cell culture media for therapeutic reprogramming of human post-natal stem cells designated PM-10 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mL DL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL rhEGF, 1 ng/mL rhFGF-2, 10 ng/mL rhGDNF and 1,000 units human LIF.

24. A cell culture media for therapeutic reprogramming of human post-natal stem cells designated PM-20 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mL DL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL rhEGF, 110 ng/mL rhFGF-2, 20 ng/mL rhGDNF and 1,000 units human LIF.

25. A cell culture media for therapeutic reprogramming of human post-natal stem cells designated PM-100 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL rhEGF, 110 ng/mL rhFGF-2, 10 ng/mL rhGDNF and 1,000 units human LIF.

26. A cell culture media for therapeutic reprogramming of human post-natal stem cells designated PM-1 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL rhEGF, 10 ng/mL rhFGF-2, 2 ng/mL rhGDNF and 1,000 units human LIF.

27. A cell culture media for therapeutic reprogramming of human post-natal stem cells designated PM-3 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 12% fetal bovine serum, 20 ng/mL rhEGF, 10 ng/mL rhFGF-2, 2 ng/mL rhGDNF and 1,000 units human LIF.

28. A cell culture media for therapeutic reprogramming of human post-natal stem cells designated PM-5 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL rhEGF, 2 ng/mL rhFGF-2, 20 ng/mL rhGDNF and 1,000 units human LIF.

29. A cell culture media for therapeutic reprogramming of human post-natal stem cells designated PM-101 consisting essentially of Stem Cell Basal Medium, 2 mM L-glutamine, MEM vitamin solution, 0.1 mM MEM non-essential amino acids, 10⁻⁴ M ascorbic acid, 10 μg/mL d-biotin, 80 ng/mL β-estradiol, 60 ng/mL progesterone, 5 mg/mL bovine albumin, 1 μL/mLDL-lactic acid, 30 μg/mL pyruvic acid, 6 mg/mL D-(+)-glucose, 30 nM sodium selenite, 60 μM putrescine, 100 μg/mL transferring, 25 μg/mL insulin, 1× penicillin/streptomycin, 10×10⁻⁵ M β-mercaptoethanol, the improvement comprising 1% fetal bovine serum, 20 ng/mL rhEGF, 10 ng/mL rhFGF-2, 10 ng/mL rhGDNF and 1,000 units human LIF.

30. A pluripotent therapeutic composition comprising a therapeutically reprogrammed human post-natal stem cell.

31. The pluripotent therapeutic composition of claim 30 wherein said human post-natal stem cell is a primordial sex cell.

32. The therapeutic reprogramming method of claim 31 wherein said primordial sex cell is a diploid germ cell isolated from a male or female post-natal source.

33. The pluripotent therapeutic composition of claim 31 wherein said primordial sex cell is a spermatogonial stem cell.

34. The pluripotent therapeutic composition of claim 30 wherein said therapeutically reprogrammed human post-natal stem cell is produced according to the therapeutic reprogramming method of claim 1.
